(19) United States
(12) Patent Application Publication
(10) Pub. No.: US 2024/0091150 A1
(43) Pub. Date: Mar. 21, 2024

(54) PHARMACEUTICAL COMPOSITION COMPRISING, AS THICKENING AGENT, A COMPOSITION HAVING POLAR MEDIA THICKENING PROPERTIES

(71) Applicant: SOCIETE D'EXPLOITATION DE PRODUITS POUR LES INDUSTRIES CHIMIQUES SEPPIC, PARIS CEDEX 07 (FR)

(72) Inventors: Miruna BODOC, Castres (FR); Stéphane MONTEILLET, Castres (FR)

(21) Appl. No.: 18/260,175

(22) PCT Filed: Dec. 22, 2021
(86) PCT No.: PCT/EP2021/087345

§ 371 Date: Jun. 30, 2023

(30) Foreign Application Priority Data
Jan. 7, 2021 (FR) 2100109

(51) Int. Cl. A61K 9/107; A61K 47/34

(52) U.S. Cl. CPC A61K 9/107; A61K 47/34

(57) ABSTRACT

A pharmaceutical composition (F) comprising at least one pharmaceutical active principle and as thickener a composition (C_{A}) in the form of an emulsion of the self-invertible water-in-oil type comprising, per 100% of its mass, a mass content of greater than or equal to 20% of a polymer (P) consisting of monomer units derived from partially or totally salified glutamic acid (GA), and of units derived from at least one crosslinking agent (XLA) bearing at least two glycidyl functions.

The present invention relates to a pharmaceutical composition (F) comprising at least one pharmaceutical active principle and, as thickener, a composition (C_{A}) in the form of an emulsion of the self-invertible water-in-oil type and to the process for preparing such a composition.

Polymers are widely used today in pharmaceutical formulations for topical use and represent the second most widely used family of products in complex formulations of this type. Pharmaceutical compositions contain polar phases, for instance phases consisting of water, and in most cases require the use of rheology modifiers, for instance polymers, to increase the viscosity of these polar phases, and also to impart well-defined rheological behavior.

Among the polymers which modify the rheology of polar phases, mention may be made of natural polymers, for instance polysaccharides based on saccharides or polysaccharides based on saccharide derivatives or else synthetic polymers, of linear or branched, crosslinked or noncrosslinked, anionic or cationic, or amphiphilic, polyelectrolyte type. Predominantly present on the market of ingredients intended for pharmaceutical formulations for topical use, synthetic polymers have the property of being deployed, in the polar phase, under the effect of electrostatic repulsions due to the presence of charges (negative and/or positive) on the linear or branched, crosslinked or noncrosslinked polymer backbone. These rheology modifiers bring both an increase in the viscosity of the polar phase, and also a certain consistency and/or a stabilizing effect to the pharmaceutical formulation for topical use.

In order to meet the needs of formulators, various recent scientific studies have reported the development of new, innovative and varied polymeric systems. Thus, polymers used in pharmacology can play a functional role as film-forming agents, rheology modifiers, agents allowing the stabilization of fatty phases in oil-in-water and water-in-oil emulsions and particle stabilization.

The polymers which modify the rheology of polar phases, more particularly of aqueous phases, are mainly polyelectrolytes, resulting from the radical polymerization of (meth)acrylic type monomers, i.e. acrylic acid, methacrylic acid, esters derived from acrylic acid or methacrylic acid, or alternatively acrylamide or methacrylamide derivatives.

Developing new biobased and biodegradable rheology modifiers, that are as efficient as the synthetic polymers currently used, still constitutes a major challenge and a key issue for suppliers of pharmaceutical ingredients. Indeed, until now the solutions mainly used for thickening polar phases involve ingredients originating from raw materials of petrochemical origin and notably acrylic acid and derivatives thereof, or methacrylic acid and derivatives thereof.

Given the growing concern of consumers for economy and sustainable development, replacing raw materials of petrochemical origin with raw materials of renewable origin to prepare polymers is a priority research area.

To date, the literature describes the use of various natural polymers or polymers from renewable raw materials, the monomer units of which come from the family of sugars (glucose, arabinose, xylose, galactose, mannose, ribose, glucuronic acid, etc.) or from the family of amino acids (glutamic acid, aspartic acid, lysine, etc.). These polymers are mostly linear or branched depending on the plant from which they come or according to their manufacturing process.

As an example of a polymer of natural origin, mention may be made of polyglutamic acid (PGA), which is currently the subject of numerous research studies. It is a predominantly linear polymer and consists of glutamic acid (GA) monomer units. Glutamic acid is an amino acid characterized by an amine function in the α position and by two carboxylic acid functions (or carboxylates depending on the pH) in the α and γ positions (cf. chemical formula No. 1).

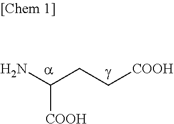

### Chemical Structure of Glutamic Acid (GA)

One of the ways to increase the branching of a synthetic or natural polymer or of a polymer of natural origin consists in performing crosslinking reactions. The purpose of crosslinking polymer chains is to connect together several polymer chains, which, when added to a polar phase, and more particularly to water, appear as a three-dimensional network that is insoluble in water, but swellable with water, then leading to the production of an aqueous gel.

The preparation of crosslinked polymers may be performed:

In one step by reacting the monomers and the crosslinking agent during the polymerization reaction, or
In at least two steps, the first of which consists in preparing the polymer, and the second consists in reacting the polymer with a crosslinking agent to obtain a crosslinked polymer.

Various reactions exist for the crosslinking of gamma-polyglutamic acid (PGA), which makes it possible to obtain polymers of natural origin with improved thickening properties in polar media, and notably in aqueous media. Among the crosslinking agents known to be used in the polyglutamic acid (PGA) crosslinking reaction, polyepoxide derivatives are the most widely described since they make it possible to perform crosslinking processes under environmentally friendly conditions (moderate temperature, reaction in aqueous media, and in the absence of harmful solvents).

However, the implementation of these processes requires diluting the (PGA) to high levels, which leads to the production of a composition in the form of an aqueous gel comprising, per 100% of its mass, a mass content less than or equal to 10% of a polymer (P), which is difficult for formulators to implement.

On this basis, a problem that arises is that of providing a user-friendly pharmaceutical composition comprising polymers of natural origin, the raw materials of which are renewable and which have thickening properties for polar media and more particularly for aqueous media.

One solution of the present invention is a pharmaceutical composition (F) comprising at least one pharmaceutical active principle and, as thickener, a composition (C_{A}) in the form of an emulsion of the self-invertible water-in-oil type comprising, per 100% of its mass, a mass content of greater than or equal to 20% of a polymer (P) consisting of monomer units derived from partially or totally salified glutamic acid (GA), and of units derived from at least one crosslinking agent (XLA) bearing at least two glycidyl functions.

For the purposes of the present invention, the term “water-in-oil type emulsion” denotes a heterogeneous mixture of two immiscible liquids, one being dispersed in the form of small droplets in the other, said mixture being thermodynamically unstable and stabilized by the presence of a surfactant system comprising at least one emulsifying surfactant.

For the purposes of the present invention, the term “emulsion of the self-invertible water-in-oil type” denotes a water-in-oil emulsion as defined above, in which the emulsifying surfactants present give the emulsion a hydrophilic-lipophilic balance (HLB) such that, once said emulsion has been added to a polar phase, for instance water, the direction of the emulsion will change from water-in-oil to oil-in-water, thereby placing the polymer (P) in contact with the polar phase to be thickened.

In the polymer (P) present in the composition (C_{A}) that is the subject of the present invention, the monomer units derived from partially or totally salified glutamic acid (GA) are linked together:

either in such a way that the amine function of a glutamic acid (GA) monomer unit is covalently linked with the carboxylic function located in the alpha a position of a second glutamic acid (GA) monomer unit; the resulting polymer is then called “α-polyglutamic acid” or PAGA (cf. chemical formula No. 2) which is partially or totally salified,

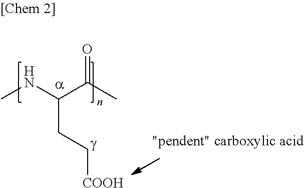

### Chemical Structure of α-Polyglutamic Acid or PAGA

or in such a way that the amine function of a glutamic acid (GA) monomer unit is covalently linked to the carboxylic function of the side chain located in the gamma (γ) position of a second glutamic acid (GA) monomer unit; the resulting polymer is then called “γ-polyglutamic acid” or PGGA (cf. chemical formula No. 3) which is partially or totally salified.

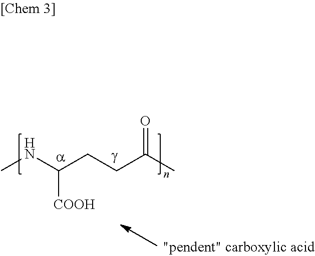

### Chemical Structure of Υ-Polyglutamic Acid or PGGA

In general, PGA can be prepared chemically according to peptide synthesis methods known to those skilled in the art, notably passing through selective protection, activation, coupling and deprotection steps. The coupling generally consists of a nucleophilic attack of the amine function of a glutamic acid monomer unit on an activated carboxylic acid function of another glutamic acid monomer unit.

PGGA can also be obtained via processes comprising at least one microbial fermentation step involving the use of at least one bacterial strain.

For the purposes of the present invention, in the polymer (P) as defined previously, the term “salified” indicates that the “pendant” carboxylic acid function present on each glutamic acid (GA) monomer unit of the polymer (in the gamma position in the case of PAGA or in the alpha position in the case of PGGA) is in an anionic or carboxylate form. The counterion of this carboxylate function is a cation derived, for instance, from alkali metal salts such as sodium, potassium or salts of nitrogenous bases such as amines, lysine or monoethanolamine (HO—CH2-CH2-NH2).

For the purposes of the present invention, the term “crosslinking agent (XLA)” denotes a chemical molecule whose structure makes it possible to bond covalently to at least two polymer chains.

For the purposes of the present invention, the term “crosslinking agent (XLA) bearing at least two glycidyl functions” denotes a crosslinking agent (XLA) as defined above, the molecular structure of which comprises at least two glycidyl units or functions of formula (I′):

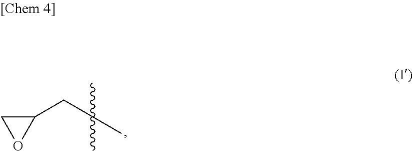 [SMILES: *CC1CO1]

The crosslinking of the polymer chains of the polymer (P) is performed according to a reaction between the terminal free amine function (—NH2) and/or one or more “pendent” or terminal carboxylic or carboxylate functions (—COOH or —COO⁻) present in the structure of said polymer (P), and at least one epoxy group present in the structure of the crosslinking agent (XLA) bearing at least two glycidyl functions.

The crosslinking agent (XLA) may be chosen from the group consisting of:

### ethylene glycol diglycidyl ether of Formula (I)

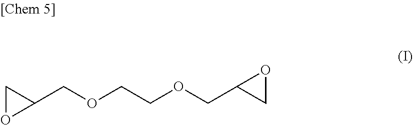 [SMILES: C(COCC1CO1)OCC1CO1]

### The Compound of Formula (II)

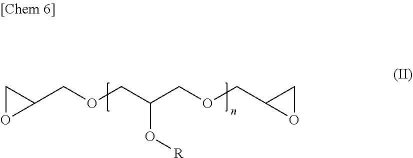

with R representing a hydrogen atom or the radical

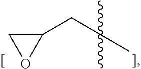 [SMILES: *CC1CO1]

and n which represents an integer greater than or equal to one and less than or equal to 10;

when R represents a hydrogen atom and n is equal to 1, the compound of formula (II) is more particularly the compound of formula (IIa) or glyceryl diglycidyl ether

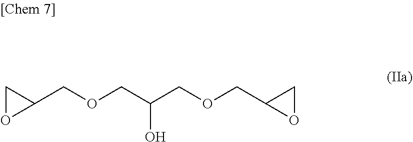 [SMILES: OC(COCC1CO1)COCC1CO1]

when R represent

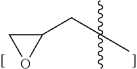 [SMILES: *CC1CO1]

and n is equal to 1, the compound of formula (II) is more particularly the compound of formula (IIb) or glyceryl triglycidyl ether

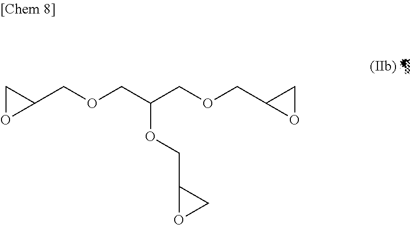 [SMILES: C(OCC1CO1)C(COCC1CO1)OCC1CO1]

when R represents a hydrogen atom and n is equal to 2, the compound of formula (II) is more particularly the compound of formula (IIc) or diglyceryl diglycidyl ether

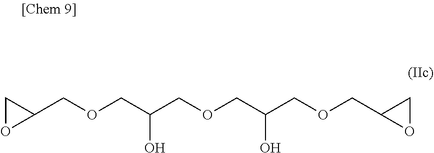 [SMILES: OC(COCC(O)COCC1CO1)COCC1CO1]

when R represents

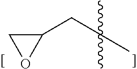 [SMILES: *CC1CO1]

and n is equal to 2, the compound of formula (II) is more particularly the compound of formula (IId) or diglyceryl tetraglycidyl ether

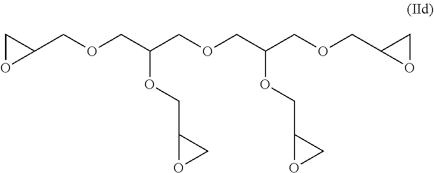 [SMILES: C(OCC(COCC1CO1)OCC1CO1)C(COCC1CO1)OCC1CO1]

### 1,3-propanediol diglycidyl ether of Formula (III)

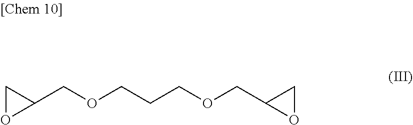 [SMILES: C(COCC1CO1)COCC1CO1]

### 1,2-propanediol diglycidyl ether of Formula (IV)

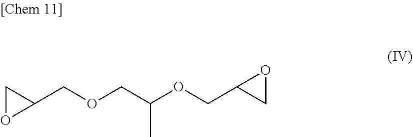

### 1,4-butanediol diglycidyl ether of Formula (V)

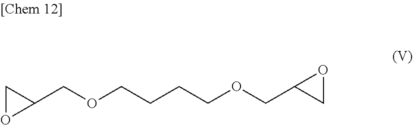 [SMILES: C(CCOCC1CO1)COCC1CO1]

### 1,2-butanediol diglycidyl ether of Formula (VI)

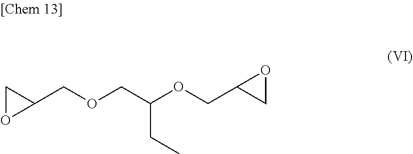

### 1,3-butanediol diglycidyl ether of Formula (VII)

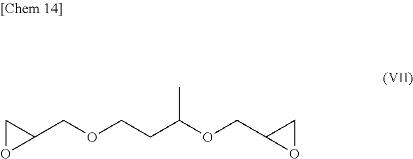

### 1,6-hexanediol diglycidyl ether of Formula (VIII)

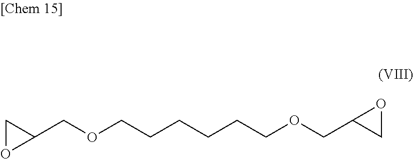 [SMILES: C(CCCOCC1CO1)CCOCC1CO1]

### The Compound of Formula (IX)

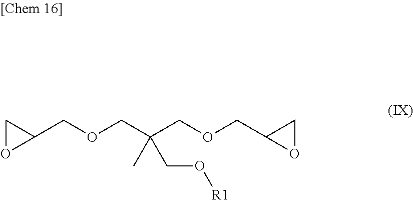

with R1 representing a hydrogen atom or

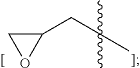 [SMILES: *CC1CO1]

when R1 represents a hydrogen atom, the compound of formula (IX) is more particularly the compound of formula (IXa) or trimethylolethane diglycidyl ether

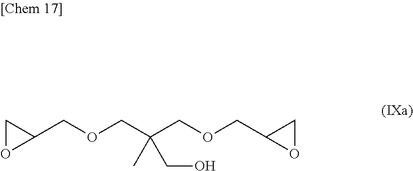 [SMILES: CC(CO)(COCC1CO1)COCC1CO1]

when R1 represents

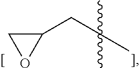 [SMILES: *CC1CO1]

the compound of formula (IX) is more particularly the compound of formula (IXb) or trimethylolethane triglycidyl ether

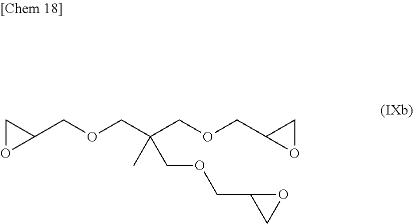 [SMILES: CC(COCC1CO1)(COCC1CO1)COCC1CO1]

### The Compound of Formula (X)

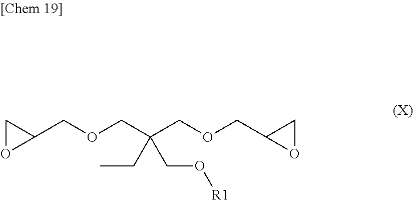

with R1 representing a hydrogen atom or the glycidyl radical

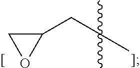 [SMILES: *CC1CO1]

when R1 represents a hydrogen atom, the compound of formula (X) is more particularly the compound of formula (Xa) or trimethylolpropane diglycidyl ether

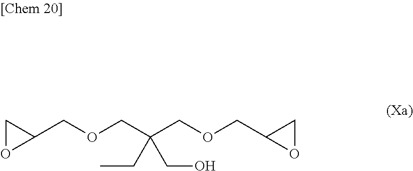 [SMILES: CCC(CO)(COCC1CO1)COCC1CO1]

when R1 represents the glycidyl radical

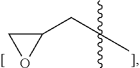 [SMILES: *CC1CO1]

the compound of formula (X) is more particularly the compound of formula (Xb) or trimethylolpropane triglycidyl ether

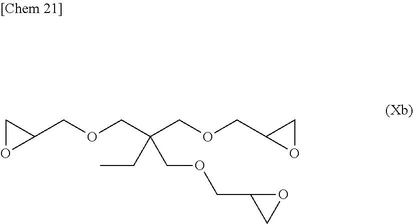 [SMILES: CCC(COCC1CO1)(COCC1CO1)COCC1CO1]

### The Compound of Formula (XI)

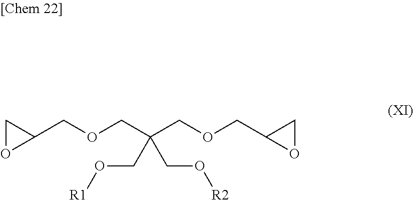

with R1 and R2, independent, which represent a hydrogen atom or the glycidyl radical

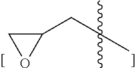 [SMILES: *CC1CO1]

when R1 and R2 each represent a hydrogen atom, the compound of formula (XI) is more particularly the compound of formula (XIa) or pentaerythrityl diglycidyl ether

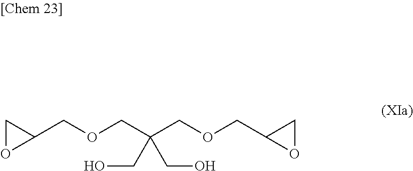 [SMILES: OCC(CO)(COCC1CO1)COCC1CO1]

when R1 represents a hydrogen atom and R2 represents the glycidyl radical

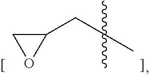 [SMILES: *CC1CO1]

the compound of formula (XI) is more particularly the compound of formula (XIb) or pentaerythrityl triglycidyl ether

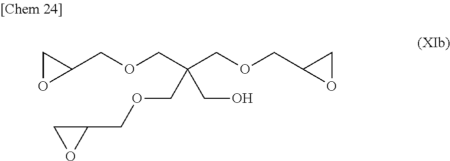 [SMILES: OCC(COCC1CO1)(COCC1CO1)COCC1CO1]

when R1 and R2 each represent the glycidyl radical

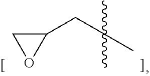 [SMILES: *CC1CO1]

the compound of formula (XI) is more particularly the compound of formula (XIc) or pentaerythrityl tetraglycidyl ether

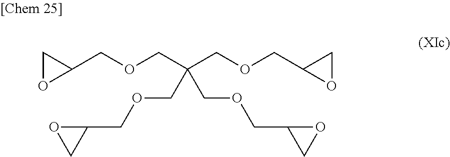 [SMILES: C(OCC(COCC1CO1)(COCC1CO1)COCC1CO1)C1CO1]

### The Compound of Formula (XII)

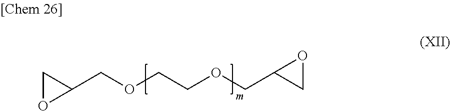

with m representing an integer greater than or equal to 2

### The Compound of Formula (XIII)

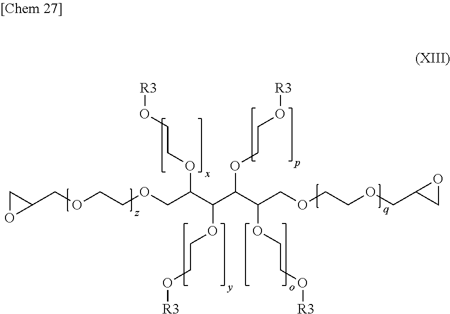

with R3 representing a hydrogen atom or

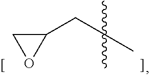 [SMILES: *CC1CO1]

and x, y, z, o, p and q, independently of each other, represent an integer greater than or equal to 2 and less than or equal to 10.

Depending on the case, the pharmaceutical composition may have one or more of the following features:
in composition (C_{A}) the mass content of the polymer (P) is greater than or equal to 20% and less than or equal to 60%;
in composition (C_{A}) the polymer (P) is gamma-polyglutamic acid in acid form, or in partially or totally salified form.
in composition (C_{A}), the polymer (P), per 100 mol % of monomer units derived from partially or totally salified glutamic acid (GA), the crosslinking agent (XLA) represents from 0.5 mol % to 20 mol %;
the composition (C_{A}) has a viscosity of between 100 mPa·s and 10 000 mPa·s (measured with a Brookfield RVT viscometer, speed 5 rpm);
the composition (C_{A}) also comprises a monomer unit derived from the compound of formula (X′):

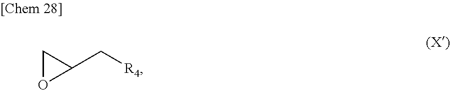

with R4 representing a linear or branched, saturated or unsaturated, functionalized or non-functionalized hydrocarbon-based radical including from 6 to 22 carbon atoms.

According to a particular aspect, R4 represents a hydrocarbon-based radical chosen from the elements of the group consisting of heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals.

According to another particular aspect, in said polymer (P), per 100% of the mass of monomer units derived from partially or totally salified glutamic acid (GA), the monomer units derived from the compound of formula (X′) represent from 1% to 50% by mass.
the pharmaceutical composition (F) comprises between 0.1% and 10% by mass of said composition (C_{A});
the pharmaceutical active principle is chosen from antibacterial agents, antimicrobial agents, antiparasitic agents, antihelminthic agents, anticoccidial agents, anti-cryptosporidian agents, anti-protozoal agents, antimycotic agents, non-steroidal anti-inflammatory agents, antiallergic and immunomodulatory agents, analgesic agents, antihistamine agents, local anesthetic agents, antisecticidal agents, antiseptic agents and antifungal agents.

By way of example:
said pharmaceutical composition (F) will comprise as pharmaceutical active agent a nonsteroidal anti-inflammatory agent and said pharmaceutical composition (F) will be for use in reducing and/or eliminating local pain, post-traumatic inflammation of joints, muscles, tendons or ligaments, localized forms of soft tissue rheumatism, localized forms of degenerative rheumatism, actinic keratosis caused by overexposure to sunlight, acute migraine, pain associated with bone metastases, fever due to malignant lymphogranulomatosis (Hodgkin's lymphoma), multi-drug resistant E. coli, Shy-Drager syndrome and diabetes mellitus.
said pharmaceutical composition (F) will comprise as pharmaceutical active agent a local anesthetic and said pharmaceutical composition (F) will be for use in treating pain, pruritus and/or anorectal disorders in humans or animals.
said pharmaceutical composition (F) will comprise as pharmaceutical active agent an antifungal agent and said pharmaceutical composition (F) will be for use in treating mycoses of the skin, scalp, mouth and/or gynecological apparatus in human or animal mammals.

A subject of the present invention is also a process for preparing a pharmaceutical composition (F) according to the invention, comprising:
a step A) of preparing the composition (C_{A}), comprising the following substeps:
a) preparation of an aqueous solution comprising partially or totally salified polyglutamic acid (PGA) with said aqueous solution comprising, per 100% of its mass, between 5% and 70% by mass of partially or totally salified PGA and a crosslinking agent (XLA) comprising at least two glycidyl functions,
b) adjustment of the pH of the aqueous solution obtained in step a) to a pH of between 3 and 11;
c) preparation of an organic phase containing at least one volatile oil, at least one other nonvolatile oil (O) and at least one emulsifying surfactant of the water-in-oil type (S₁);
d) pre-emulsification by adding the organic phase obtained in step c) to the aqueous solution obtained in step b) with stirring;
e) emulsification of the pre-emulsion obtained in step d) by homogenization with stirring;
f) distillation of the water and volatile oil contained in the emulsion obtained in step e);
g) addition of at least one emulsifying surfactant of the oil-in-water type (S₂) so as to obtain the composition (C_{A}).
A step B) of mixing at least one composition (C_{A}) prepared in step A) with at least one pharmaceutical active agent and at least one pharmaceutically acceptable medium, for instance water.

Depending on the case, the process according to the invention may have one or more of the features below:
in step a) the polyglutamic acid (PGA) is gamma-polyglutamic acid (PGGA);
in step a) all of the monomer units constituting gamma-polyglutamic acid (PGGA) are derived from sodium glutamate, potassium glutamate, ammonium glutamate, calcium glutamate, magnesium glutamate or a mixture of these forms;
in step a) the crosslinking agent (XLA) is present in mass proportions of between 0.5% and 10% by mass relative to the mass of polyglutamic acid (PGA);
the crosslinking agent (XLA) is chosen from the members of the group consisting of the compounds of formulae (I), (II), (IIa), (IIb), (IIc), (III), (IV), (V), (VI), (VII), (VIII), (IX), (IXa), (IXb), (X), (Xa), (Xb), (XI), (XIa), (XIb), (Xc), (XII) and (XIII);
in step c) the at least emulsifying agent of the water-in-oil type (S₁) is chosen from the elements of the group consisting of sorbitan esters, polyglycerol esters, alkoxylated polyglycerol esters, polyglycol polyhydroxystearates, polyglyceryl polyhydroxystearates and alkoxylated polyglyceryl polyhydroxystearates;
in step c) the organic solution comprises, per 100% of its own mass, between 10% and 30% by mass of at least one emulsifying agent of the water-in-oil type (S₁), preferably between 15% and 20% by mass;
in step c) the emulsifying agent of the water-in-oil type (S₁) is a polyglyceryl polyhydroxystearate;
in step g) the at least emulsifying surfactant of the oil-in-water type (S₂) is chosen from members of the group consisting of a polyethoxylated fatty alcohol, a polyethoxylated hexitan ester, an alkylpolyglycoside, a composition of alkylpolyglycoside and of fatty alcohols, a polyglycerol ester, a composition of polyglycerol ester and of polyglycerol;
step d) is performed so that the mass ratio between the aqueous solution and the organic phase is between 90/10 and 10/90, preferably between 20/80 and 40/60;
in step a), the aqueous solution also comprises at least one compound of formula (X′):

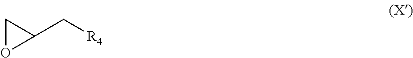

with R4 representing a linear or branched, saturated or unsaturated, functionalized or non-functionalized hydrocarbon-based radical including from 6 to 22 carbon atoms.

According to a particular aspect, R4 represents a hydrocarbon-based radical chosen from the elements of the group consisting of heptyl, octyl, nonyl, decyl, undecyl, undecenyl, dodecyl, tridecyl, tetradecyl, pentadecyl, hexadecyl, heptadecyl, octadecyl, hydroxyoctadecyl, oleyl, linoleyl, linolenyl, eicosyl and dodecosyl radicals.

According to another particular aspect, the content of compound of formula (X′) in the polar solution is, per 100% by mass of said aqueous solution, between 0.05% and 35% by mass, it being understood that the sum of the mass proportions of the polymer (P), of the crosslinking agent (XLA), of the water and of the compound of formula (X′) is equal to 100%.
in step e) the homogenization is performed under mechanical shear stirring.
in step f) the distillation is performed under vacuum and with heating. This has the effect of crosslinking the polyglutamic acid and concentrating the emulsion;
in steps c) and f) the volatile oil is a light isoparaffin including 8 to 11 carbon atoms. This isoparaffin may be chosen from those sold under the names Isopar™ G, Isopar™ L, Isopar™ H and Isopar™ J.

The choice of a concentrated inverse emulsion process makes it possible to dissolve the starting poly-gamma-glutamic acid (PGGA), its possible co-constituents, and also the crosslinking agent(s) in the aqueous phase of the emulsion. The production of the emulsion makes it possible to create droplets isolated from each other, enabling the crosslinking of the PGA without caking of the reaction medium due to the increase in viscosity of the aqueous phase during the crosslinking step. The concentration step by distillation of a light fatty phase leads to the production of a product in liquid form with an active material content of greater than 20%.

According to a particular aspect, in the composition (C_{A}) which is the subject of the present invention, the mass content of the polymer (P) is greater than or equal to 20% and less than or equal to 60%; and more particularly greater than or equal to 20% and less than or equal to 40%.

PGGA can exist in different conformational forms in solution in water. These forms depend on the inter- and intra-molecular hydrogen bonds and thus on the pH, the polymer concentration, the ionic strength of the solution, and also the temperature. The chains of the PGGA can thus adopt an a helical shape, a β sheet of aggregates or else be in a disordered and random state.

According to a particular aspect, in the composition (C_{A}) which is the subject of the present invention, the polymer (P) is in a helical conformation when it is present in a solution at a mass content of less than or equal to 0.1% and of which said solution has a pH value of less than or equal to 7.

According to a particular aspect, in the composition (C_{A}) which is the subject of the present invention, the polymer (P) is in sheet conformation when it is present in a solution at a mass content of less than or equal to 0.1% and of which said solution has a pH value above 7.

According to a particular aspect of the composition (C_{A}) which is the subject of the present invention, in the polymer (P), per 100 mol % of monomer units derived from partially or totally salified glutamic acid (GA), the crosslinking agent (XLA) represents from 1 mol % to 20 mol %, and even more particularly from 1 mol % to 18 mol %.

According to another particular aspect, the composition (C_{A}) has a viscosity of between 1000 mPa·s and 10 000 mPa·s (measured with a Brookfield RVT viscometer, speed 5 rpm), more particularly between 1000 mPa·s and 5000 mPa·s.

According to another particular aspect, the crosslinking agent (XLA) is ethylene glycol diglycidyl ether of formula (I).

According to another particular aspect, in step a) of the process which is the subject of the present invention, the partially or totally salified polyglutamic acid (PGA) is in the form of a potassium, sodium or ammonium salt, and more particularly in the form of a sodium salt.

According to another particular aspect, in step a) of the process which is the subject of the present invention, the aqueous solution comprises, per 100% of its mass, between 5% and 60% by mass, more particularly between 10% and 50% by mass, of partially or totally salified polyglutamic acid (PGA).

According to another particular aspect, in step a) of the process which is the subject of the present invention, the crosslinking agent (XLA) is chosen from at least one of the members of the group consisting of the compounds of formulae (I), (II), (III), (IV), (V), (VI), (VII), (VIII), (IX), (X), (XI), (XII) and (XIII) as defined previously.

According to another aspect, in step c) of the process which is the subject of the present invention, the term “volatile oil” denotes a fatty substance which is liquid at a temperature of 25° C. at atmospheric pressure, and whose flash point is between 40° C. and 100° C.

According to a more particular aspect, for the purposes of the present invention, the term “volatile oil” means an element of the group consisting of branched alkanes, including from seven to forty carbon atoms, such as isododecane, isopentadecane, isohexadecane, isoheptadecane, isooctadecane, isononadecane or isoeicosane), or mixtures of some of them such as those mentioned below and identified by their INCI name: C7-8 isoparaffin, C8-9 isoparaffin, C9-11 isoparaffin, C9-12 isoparaffin, C9-13 isoparaffin, C9-14 isoparaffin, C9-16 isoparaffin, C10-11 isoparaffin, C10-12 isoparaffin, C10-13 isoparaffin, C11-12 isoparaffin, C11-13 isoparaffin, and C11-14 isoparaffin.

According to an even more particular aspect, for the purposes of the present invention, the term “volatile oil” means at least one element of the group consisting of isododecane, isohexadecane, C7-8 isoparaffin, C8-9 isoparaffin, C9 -11 isoparaffin, C11-13 isoparaffin and C11-14 isoparaffin.

According to another even more particular aspect of the present invention, the volatile oil is chosen from an element of the group consisting of C8-9 isoparaffin, C9-11 isoparaffin, C11-13 isoparaffin and C11-14 isoparaffin.

According to another even more particular aspect of the present invention, the “volatile oil” is chosen from an element of the group consisting of the isoparaffins sold under the brand names Isopar™ G, Isopar™ L, Isopar™ H or Isopar™ J.

According to another aspect, in step c) of the process which is the subject of the present invention, the term “oil (0)” denotes a fatty substance that is liquid at a temperature of 25° C. at atmospheric pressure, notably:
linear alkanes including from 11 to 19 carbon atoms;
branched alkanes including from 11 to 40 carbon atoms, such as isododecane, isopentadecane, isohexadecane, isoheptadecane, isooctadecane, isononadecane or isoeicosane, or mixtures of certain thereof such as those mentioned hereinbelow and identified by their INCI name: C12-14 isoparaffin, C12-20 isoparaffin, C13-14 isoparaffin, C13-16 isoparaffin.
cycloalkanes optionally substituted with one or more linear or branched alkyl radicals;
white mineral oils, such as those sold under the following names:

Marcol™ 52, Marcol™ 82, Drakeol™ 6VR, Eolane™ 130, Eolane™ 150, Hemisqualane (or 2,6,10-trimethyldodecane; CAS number: 3891-98-3), squalane (or 2,6,10,15,19,23-hexamethyltetracosane), hydrogenated polyisobutene or hydrogenated polydecene;
Mixtures of alkanes including from 15 to 19 carbon atoms, said alkanes being linear alkanes, branched alkanes and cycloalkanes, and more particularly the mixture (M1) which comprises, per 100% of its mass:
a mass proportion of branched alkanes of greater than or equal to 90% and less than or equal to 100%,
a mass proportion of linear alkanes of greater than or equal to 0% and less than or equal to 9%,
a mass proportion of cycloalkanes of greater than or equal to 0% and less than or equal to 1%, and

more particularly said mixture (M1) is characterized in that it comprises, per 100% of its mass:
a mass proportion of greater than or equal to 95% of branched alkanes, linear alkanes and cycloalkanes and less than or equal to 100% including from 15 to 19 carbon atoms, and
a mass proportion of greater than or equal to 0% and less than or equal to 5% of branched alkanes, linear alkanes and cycloalkanes including less than 14 carbon atoms, and linear alkanes and cycloalkanes including more than 20 carbon.

For the purposes of the present invention, the term “linear alkanes” present in the mixture (M1) as defined above, and including from 15 to 19 carbon atoms, more particularly means elements chosen from the group consisting of n-pentadecane, n-hexadecane, n-heptadecane, n-octadecane and n-nonadecane.

For the purposes of the present invention, the term “branched alkanes” present in the mixture (M1) as defined above, and including from 15 to 19 carbon atoms, more particularly means elements chosen from the group consisting of isopentadecane, isohexadecane, isoheptadecane, isooctadecane and isononadecane.

The mixture (M1) is more particularly the mixture sold under the brand name Emogreen™ L15 or else the mixture sold under the brand name Emogreen™ L19.
The fatty alcohol ethers of formula (XIV):

Z1-O—Z2 (XIV),

in which Z1 and Z2, which may be identical or different, represent a linear or branched alkyl radical including from 5 to 18 carbon atoms, for example dioctyl ether, didecyl ether, didodecyl ether, dodecyl octyl ether, dihexadecyl ether, (1,3-dimethylbutyl) tetradecyl ether, (1,3-dimethylbutyl) hexadecyl ether, bis(1,3-dimethylbutyl) ether or dihexyl ether;
the monoesters of fatty acids and alcohols of formula (XV):

R′1-(C═O)—O—R′2 (XV),

in which R′1-(C═O) represents a saturated or unsaturated, linear or branched acyl radical including from 8 to 24 carbon atoms, and R′2 represents, independently of R′1, a saturated or unsaturated, linear or branched hydrocarbon-based chain including from 1 to 24 carbon atoms, for example methyl laurate, ethyl laurate, propyl laurate, isopropyl laurate, butyl laurate, 2-butyl laurate, hexyl laurate, methyl cocoate, ethyl cocoate, propyl cocoate, isopropyl cocoate, butyl cocoate, 2-butyl cocoate, hexyl cocoate, methyl myristate, ethyl myristate, propyl myristate, isopropyl myristate, butyl myristate, 2-butyl myristate, hexyl myristate, octyl myristate, methyl palmitate, ethyl palmitate, propyl palmitate, isopropyl palmitate, butyl palmitate, 2-butyl palmitate, hexyl palmitate, octyl palmitate, methyl oleate, ethyl oleate, propyl oleate, isopropyl oleate, butyl oleate, 2-butyl oleate, hexyl oleate, octyl oleate, methyl stearate, ethyl stearate, propyl stearate, isopropyl stearate, butyl stearate, 2-butyl stearate, hexyl stearate, octyl stearate, methyl isostearate, ethyl isostearate, propyl isostearate, isopropyl isostearate, butyl isostearate, 2-butyl isostearate, hexyl isostearate, isostearyl isostearate;
the diesters of fatty acids and glycerol of formulae (XVI) and (XVII):

R′3-(C═O)—O—CH2-CH(OH)—CH2-O—(C═O)—R′4 (XVI)

R′5-(C═O)—O—CH2-CH[O—(C═O)—R′6]-CH2-OH (XVII),

in which R′3-(C═O) and R′4-(C═O), R′5-(C═O), R′6-(C═O), which may be identical or different, represent a saturated or unsaturated, linear or branched acyl group including from eight to twenty-four carbon atoms.
the triesters of fatty acids and of glycerol of formula (XVIII):

R′7-(C═O)—O—CH2-CH[O—(C═O)—R″8]-CH2-O—(C═O)—R″9 (XVIII),

in which R′7-(C═O), R′8-(C═O) and R′9-(C═O), which may be identical or different, represent a saturated or unsaturated, linear or branched acyl group including from 8 to 24 carbon atoms.

According to another particular aspect of the present invention, said oil (O) is chosen from:
undecane, tridecane, isododecane or isohexadecane;
Mixtures of alkanes and isoalkanes and cycloalkanes such as the mixture (M1) as defined previously and the mixtures sold under the names Emogreen™ L15, Emogreen™ L19, Emosmart™ L15, Emosmart™ L19, Emosmart™ V21 and Isopar™ M;
The white mineral oils sold under the names Marcol™ 52, Marcol™ 82,
Drakeol™ 6VR, Eolane™ 130 or Eolane™ 150;
hemisqualane, squalane, hydrogenated polyisobutene or hydrogenated polydecene;
dioctyl ether or didecyl ether;
isopropyl myristate, hexyl palmitate, octyl palmitate, isostearyl isostearate, octanoyl/decanoyl triglyceride, hexadecanoyl/octadecanoyl triglyceride, and triglycerides derived from rapeseed oil, sunflower oil, linseed oil or palm oil.

According to another aspect, in step c) of the process that is the subject of the present invention, the term “emulsifying surfactant of the water-in-oil type (S1)” denotes an emulsifying surfactant having an HLB value (Hydrophilic-Lipophilic Balance) that is low enough to induce the formation of a water-in-oil type emulsion, namely an emulsion in which the aqueous phase will be dispersed and stabilized in the oily organic phase.

As emulsifying surfactant of water-in-oil type, examples that may be mentioned include anhydrohexitol esters of linear or branched, saturated or unsaturated aliphatic carboxylic acids, including from 12 to 22 carbon atoms, optionally substituted with one or more hydroxyl groups, and more particularly esters of anhydrohexitols chosen from anhydrosorbitols and anhydromannitols and of linear or branched, saturated or unsaturated aliphatic carboxylic acids including from 12 to 22 carbon atoms, optionally substituted with one or more hydroxyl groups.

In step c) of the process that is the subject of the present invention, the emulsifying system (S1) of the water-in-oil type is more particularly chosen from the elements of the group consisting of
sorbitan laurate, for example the product sold under the name Montane™ 20,
sorbitan palmitate, for example the product sold under the name Montane™ 40,
sorbitan stearate, for example the product sold under the name Montane™ 60,
sorbitan oleate, for example the product sold under the name Montane™ 80,
sorbitan sesquioleate, for example the product sold under the name Montane™ 83,
sorbitan trioleate, for example the product sold under the name Montane™ 85,
sorbitan isolaurate,
sorbitan isostearate, for example the product sold under the name Montane™ 70,
mannitan laurate, mannitan oleate, or a mixture of these esters; polyesters with a molecular weight of between 1000 and 3000 g/mol and resulting from condensation between a poly(isobutenyl)succinic acid or its anhydride, such as Hypermer™ 2296, or the mixture sold under the brand name Simaline™ IE 501 A.

As emulsifying surfactant of water-in-oil type (S1), mention may be made, for example, of polyglycerol esters, a compound of formula (XIX):

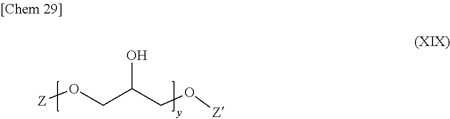

in which Z represents an acyl radical of formula R2-C(═O)—, in which R2 represents a saturated or unsaturated, linear or branched, aliphatic hydrocarbon-based radical, comprising from 11 to 35 carbon atoms, and more particularly a radical chosen from the dodecanoyl, tetradecanoyl, hexadecanoyl, octadecanoyl, eicosanoyl, docosanoyl, oleyl, linoleyl, linolenoyl or isostearyl radicals, Z′ represents the acyl radical of formula R2-C(═O)— as defined above, with Z′ identical to or different from Z, or a hydrogen atom, and y represents an integer greater than or equal to 2 and less than or equal to 20.

According to a more particular aspect, the compound of formula (XIX) is chosen from the elements of the group consisting of decaglyceryl oleate, decaglyceryl isostearate, decaglyceryl monolaurate, decaglyceryl monolinoleate and decaglyceryl monomyristate.

As emulsifying surfactant of water-in-oil type (S1), mention may be made, for example, of alkoxylated polyglycerol esters, a compound of formula (XX):

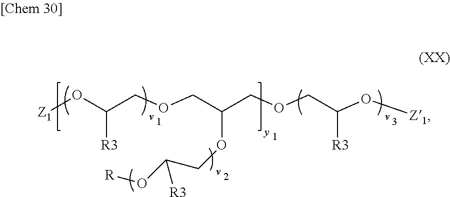

in which Z1 represents an acyl radical of formula R′2-C(═O)—, in which R′2 represents an aliphatic hydrocarbon-based radical, saturated or unsaturated, linear or branched, containing from 11 to 35 carbon atoms, and more particularly a radical chosen from the dodecanoyl, tetradecanoyl, hexadecanoyl, octadecanoyl, eicosanoyl, docosanoyl, oleyl, linoleyl, linolenoyl or isostearyl radicals, Z1′ represents the acyl radical of formula R′2-C(═O)— as defined above above, with Z1′ identical to or different from Z1, or a hydrogen atom, R3 represents a hydrogen atom, a methyl radical, or an ethyl radical, y1 represents an integer greater than or equal to 2 and less or equal to 20, v1, v2, v3, identical or different, represent an integer greater than or equal to 0 and less than or equal to 50, and the sum [(y1·v1)+(y1·v2)+v3)] is a whole number greater than or equal to 1 and less than or equal to 50.

As emulsifying surfactant of the water-in-oil type (S1), mention may be made, for example, of the polyglycol polyhydroxystearates of formula (XXI):

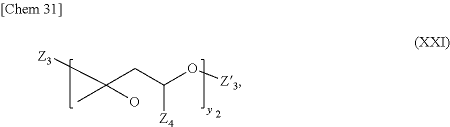

in which formula (XXI) y2 represents an integer greater than or equal to 2 and less than or equal to 50, Z4 represents a hydrogen atom, a methyl radical or an ethyl radical, Z3 represents a radical of formula (XXII):

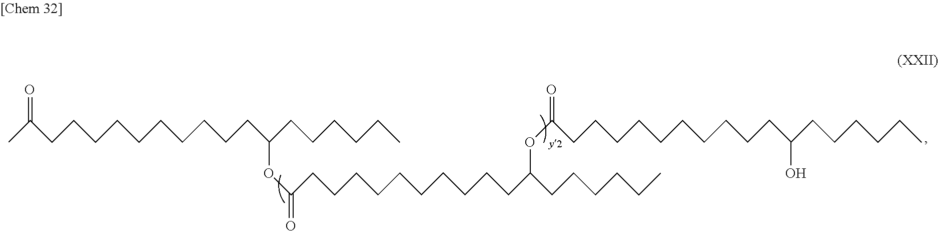

in which formula (XXII) y′2 represents an integer greater than or equal to 0 and less than or equal to 10, more particularly greater than or equal to 1 and less than or equal to 10 and Z′3 represents a radical of formula (XXII) as defined above, with Z3′ identical to or different from Z3, or a hydrogen atom.

Examples of emulsifying surfactant of water-in-oil type of formula (XXI) that may be used for preparing the emulsifying (S1) system include PEG-30 dipolyhydroxystearate, sold under the name Simaline™ WO, or else the mixtures comprising PEG-30 dipolyhydroxystearate and sold under the names Simaline™ IE 201 A and Simaline™ IE 201 B, or else the mixture comprising trimethylolpropane-30 tripolyhydroxystearate sold under the name Simaline™ IE 301 B.

As emulsifying surfactant of the water-in-oil type (S1), mention may be made, for example, of the polyglyceryl polyhydroxystearates represented by formula (XXIII):

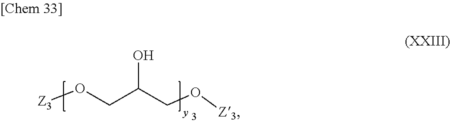

in which Z3 represents a radical of formula (XXIII) as defined above, Z′3 represents a radical of formula (XXII) as defined above, with Z3′ identical to or different from Z3, or a hydrogen atom, and y3 represents an integer greater than or equal to 2 and less than or equal to 20.

As emulsifying surfactant of the water-in-oil type (S1), mention may be made, for example, of alkoxylated polyglyceryl polyhydroxystearates, a compound represented by formula (XXIV):

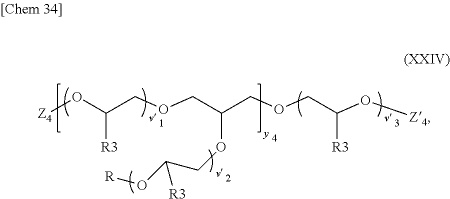

in which Z4 represents a radical of formula (XXII) as defined above, Z′4 represents a radical of formula (XXII) as defined above, with Z4′ identical to or different from Z4, or a hydrogen atom, y4 represents an integer greater than or equal to 2 and less than or equal to 20, v′1, v′2, v′3, which may be identical or different, represent an integer greater than or equal to 0 and less than or equal to 50, and the sum [(y4·v′1)+(y4·v′2)+v′3)] is an integer greater than or equal to 1 and less than or equal to 50.

According to another aspect, in step g) of the process which is the subject of the present invention, the term “emulsifying surfactant of the oil-in-water type (S2)” denotes an emulsifying surfactant having a sufficiently high HLB value to induce the formation of an emulsion of the oil-in-water type, namely an emulsion in which the oily organic phase will be dispersed and stabilized in the aqueous phase.

According to another aspect, in step g) of the process which is the subject of the present invention, as surfactant of the oil-in-water type (S2), mention may be made of the “polyethoxylated fatty alcohols” denoted by the compounds of formula (XXV):

R″—O—(CH2-CH2-O)_{n′}—OH (XXV),

with R″ representing a linear or branched, saturated or unsaturated hydrocarbon-based radical, which may bear hydroxyl groups, and including from six to twenty-two carbon atoms, and with n′ representing an integer greater than or equal to four and less than or equal to one hundred.

According to a more particular aspect, in formula (XXV), R″ represents a linear or branched, saturated hydrocarbon-based radical including from ten to twenty-two carbon atoms.

According to an even more particular aspect, the compound of formula (XXV) is a linear decyl alcohol ethoxylated with six moles of ethylene oxide, a linear decyl alcohol ethoxylated with eight moles of ethylene oxide, a linear lauryl alcohol ethoxylated with six moles of ethylene oxide, a linear lauryl alcohol ethoxylated with seven moles of ethylene oxide, a linear lauryl alcohol ethoxylated with eight moles of ethylene oxide, a linear tridecyl alcohol ethoxylated with six moles of ethylene oxide, a linear tridecyl alcohol ethoxylated with eight moles of ethylene oxide, a linear tridecyl alcohol ethoxylated with nine moles of ethylene oxide.

According to another aspect, in step g) of the process which is the subject of the present invention, as surfactant of oil-in-water type (S2), mention may be made of polyethoxylated hexitan esters, and particularly polyethoxylated sorbitan esters, the aliphatic hydrocarbon-based chain of which contains from 12 to 22 carbon atoms and in which the number of ethylene oxide units is between 5 and 40, for example sorbitan oleate ethoxylated with 20 mol of ethylene oxide, sold under the trade name Montanox™ 80, or sorbitan laurate ethoxylated with 20 mol of ethylene oxide, sold under the trade name Montanox™ 20.

According to another aspect, in step g) of the process which is the subject of the present invention, as surfactant of the oil-in-water type (S2), mention may be made of the alkyl polyglycosides compositions (C1) represented by formula (XXVI):

R″1-O-(G)x-H (XXVI)

in which x, or the average degree of polymerization, represents a decimal number between 1.05 and 5, G represents a reducing sugar residue, and R″1 represents a saturated or unsaturated, linear or branched aliphatic hydrocarbon-based radical, optionally substituted with one or more hydroxyl groups, including from 12 to 36 carbon atoms, said composition (C1) consisting of a mixture of compounds represented by the formulae (XXVI1), (XXVI2), (XXVI3), (XXVI4) and (XXVI5):

R″1-O-(G)1-H (XXVI1)

R″1-O-(G)2-H (XXVI2)

R″1-O-(G)3-H (XXVI3)

R″1-O-(G)4-H (XXVI4)

R″1-O-(G)5-H (XXVI5)

in the respective molar proportions a1, a2, a3, a4 and a5, such that:
the sum a1+a2+a3+a4+a5 is equal to 1, and that
the sum a1+2a2+3a3+4a4+5a5 is equal to x.

The term “saturated or unsaturated, linear or branched aliphatic hydrocarbon-based radical including from 12 to 36 carbon atoms, optionally substituted with one or more hydroxyl groups” denotes, for the radical R″1 in formula (XXVI) as defined above more particularly the n-dodecyl radical, the n-tetradecyl radical, the n-hexadecyl radical, the n-octadecyl radical, the n-eicosyl radical, the n-docosyl radical or the 12-hydroxyoctadecyl radical.

The term “reducing sugar” in the definition of formula (XXVI) as defined above denotes saccharide derivatives that do not have in their structures any glycoside bonds established between an anomeric carbon and the oxygen of an acetal group as defined in the reference publication: “Biochemistry”, Daniel Voet/Judith G. Voet, page 250, John Wiley & Sons, 1990. The oligomeric structure (G)ₓ may exist in any isomeric form, whether it is optical isomerism, geometrical isomerism or regioisomerism; it may also represent a mixture of isomers.

In formula (XXVI) as defined above, the group R₁—O— is linked to G via the anomeric carbon of the saccharide residue, so as to form an acetal function.

According to a particular aspect in the definition of formula (XXVI) as defined above, G represents a reducing sugar residue chosen from glucose, dextrose, sucrose, fructose, idose, gulose, galactose, maltose, isomaltose, maltotriose, lactose, cellobiose, mannose, ribose, xylose, arabinose, lyxose, allose, altrose, dextran and tallose; and more particularly, G represents a reducing sugar residue chosen from glucose, xylose and arabinose residues.

According to an even more particular aspect, in the definition of formula (XXVI), x, or the mean degree of polymerization, represents a decimal number greater than or equal to 1.05 and less than or equal to 2.5, more particularly greater than or equal to 1.05 and less than or equal to 2.0 and even more particularly greater than or equal to 1.25 and less than or equal to 2.0.

According to another aspect, in step g) of the process which is the subject of the present invention, as surfactant of the oil-in-water type (S2), mention may be made of the compositions (C2) comprising, per 100% of their mass:
from 10% to 50% by mass, more particularly from 15% to 40% by mass and even more particularly from 20% to 30% by mass of at least one composition (C1) represented by formula (XXVI) as defined previously,
from 90% to 50% by mass, more particularly from 85% to 60% by mass, and even more particularly from 80% to 70% by mass, of at least one fatty alcohol of formula (XXVII):

R′″1-OH (XXVII),
in which R″′1, which may be identical to or different from R″1, represents a saturated or unsaturated, linear or branched aliphatic hydrocarbon-based radical, optionally substituted with one or more hydroxyl groups, including from 12 to 36 carbon atoms and preferably from 12 to 22 carbon atoms.

As emulsifying surfactant of the oil-in-water type (S2), mention may be made, for example, of the polyglycerol esters of formula (XXVIII):

R12-(C═O)—[O—CH2-CH(OH)—CH2]p12-OH (XXVIII),

in which formula (XVIII) p12 represents an integer greater than or equal to one and less than or equal to fifteen; and in which the group R1-(C═O)— represents a saturated or unsaturated, linear or branched aliphatic radical including from six to twenty-two carbon atoms.

As emulsifying surfactant of the oil-in-water type (S2), mention may be made, for example, of the compositions (C13) comprising, per 100% of their masses:
from 10% by mass to 60% by mass of at least one compound of formula (XXIX):

HO—[CH2-CH(OH)—CH2-O]n12-H (XXIX)

in which formula (I) n12 represents an integer greater than or equal to one and less than or equal to fifteen; and
from 40% by mass to 90% by mass of at least one compound of formula (XXVIII) as defined previously.

Finally, a subject of the invention is also the use of said composition (C_{A}) as defined previously, as a thickening and/or emulsifying and/or stabilizing agent for a liquid aqueous pharmaceutical composition for topical use.

According to a particular aspect, said use consists in thickening polar phases, for instance aqueous, alcoholic or aqueous-alcoholic phases or polar phases comprising polyols such as glycerol.

According to another particular aspect, said use consists in stabilizing an emulsion of oil-in-water type, or of water-in-oil type, giving said emulsion a homogeneous appearance during storage under various conditions, and more particularly at 25° C. for a time at least equal to one month, and more particularly at 4° C. for a time at least equal to one month, and more particularly at 45° C. for a time at least equal to one month.

According to another particular aspect, said use consists in stabilizing solid particles in pharmaceutical compositions (F) for topical use.

These solid particles to be suspended may have various regular or irregular geometries, and may be in the form of pearls, beads, rods, flakes, leaflets or polyhedra. These solid particles are characterized by an apparent mean diameter of between 1 μm and 5 mm, more particularly between 10 μm and 1 mm.

The solid particles that may be suspended and stabilized with the polymer (P) as defined previously in pharmaceutical compositions for topical use include micas, iron oxide, titanium oxide, zinc oxide, aluminum oxide, talc, silica, kaolin, clays, boron nitride, calcium carbonate, magnesium carbonate, magnesium hydrogen carbonate, inorganic colored pigments, polyamides, such as Nylon-6, polyethylenes, polypropylenes, polystyrenes, polyesters, acrylic or methacrylic polymers, such as polymethyl methacrylates, polytetrafluoroethylene, crystalline or microcrystalline waxes, porous spheres, selenium sulfide, zinc pyrithione, starches, alginates, plant fibers, loofah particles and sponge particles.

Said pharmaceutical composition (F) for topical use, which is the subject of the present invention, is notably in the form of an aqueous solution, an emulsion or a microemulsion with a continuous aqueous phase, an emulsion or a microemulsion with an oily continuous phase, an aqueous gel, a foam, or in the form of an aerosol. It may be applied directly to the surface of the skin or else via any type of support intended to be placed in contact with the surface of the skin (paper, wipe, textile).

In general, said pharmaceutical composition for topical use (F) which is the subject of the present invention, also includes at least one or more auxiliary compounds chosen from fatty phases, foaming and/or detergent surfactants, thickening and/or gelling surfactants, thickening and/or gelling agents, stabilizers, film-forming compounds, solvents and cosolvents, hydrotropic agents, plasticizers, opacifiers, nacreous agents, superfatting agents, sequestrants, chelating agents, antioxidants, fragrances, essential oils, preserving agents, conditioning agents and deodorants.

In general, the pharmaceutical for topical use (F) according to the invention may comprise excipients and/or active principles usually used in the field of formulations for topical use, in particular pharmaceutical or dermopharmaceutical formulations.

As regards the auxiliary compounds, among the foaming and/or detergent anionic surfactants that may be combined with the pharmaceutical composition (F) for topical use according to the invention, mention may be made of alkali metal salts, alkaline-earth metal salts, ammonium salts, amine salts or amino alcohol salts of alkyl ether sulfates, of alkyl sulfates, of alkylamido ether sulfates, of alkylaryl polyether sulfates, of monoglyceride sulfates, of alpha-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkyl sulfosuccinates, of alkyl ether sulfosuccinates, of alkylamide sulfosuccinates, of alkyl sulfoacetates, of alkyl sarcosinates, of acyl isethionates, of N-acyl taurates, of acyl lactylates, of N-acylated derivatives of amino acids, of N-acylated derivatives of peptides, of N-acylated derivatives of proteins, or of fatty acids.

Among the foaming and/or detergent amphoteric surfactants optionally present in the pharmaceutical composition (F) for topical use according to the invention, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants optionally present in the pharmaceutical composition (F) for topical use according to the invention, mention may be made in particular of quaternary ammonium derivatives.

Among the foaming and/or detergent nonionic surfactants optionally present in composition (F) for topical use according to the invention, mention may be made more particularly of alkylpolyglycosides including a linear or branched, saturated or unsaturated aliphatic radical and including from 8 to 12 carbon atoms; castor oil derivatives, polysorbates, coconut kernel amides and N-alkylamines.

As examples of thickening and/or gelling surfactants optionally present in the pharmaceutical composition (F) for topical use according to the invention, mention may be made of:
optionally alkoxylated fatty esters of alkylpolyglycosides, and most particularly ethoxylated esters of methylpolyglucoside such as PEG 120 methyl glucose trioleate and PEG 120 methyl glucose dioleate sold, respectively, under the names Glucamate™ LT and Glumate™ DOE120;
alkoxylated fatty esters, such as PEG 150 pentaerythrityl tetrastearate, sold under the name Crothix™ DS53, or PEG 55 propylene glycol oleate, sold under the name Antil™ 141;
carbamates of polyalkylene glycols comprising fatty chains, such as PPG 14 laureth isophoryl dicarbamate, sold under the name Elfacos™ T211, or PPG 14 palmeth 60 hexyl dicarbamate, sold under the name Elfacos™ GT2125.

As examples of emulsifying surfactants optionally present in the pharmaceutical composition (F) for topical use according to the invention, mention may be made of nonionic surfactants, anionic surfactants and cationic surfactants.

As examples of emulsifying nonionic surfactants optionally present in the composition (F) for topical use according to the invention, mention may be made of ethoxylated castor oil and ethoxylated hydrogenated castor oil, for example the product sold under the name Simulsol™ 989; compositions comprising glycerol stearate and stearic acid poly(ethoxylated) with between 5 mol and 150 mol of ethylene oxide, for example the composition comprising stearic acid (ethoxylated) with 135 mol of ethylene oxide and glycerol stearate sold under the name Simulsol™ 165; ethoxylated sorbitan esters, for example the products sold under the name Montanox™; ethoxylated mannitan esters; sucrose esters; methyl glucoside esters.

As examples of emulsifying anionic surfactants optionally present in the cosmetic composition (F) for topical use which is a subject of the present invention, mention may be made of decyl phosphate, cetyl phosphate sold under the name Amphisol™, glyceryl stearate citrate; cetearyl sulfate; the arachidyl/behenyl phosphates and arachidyl/behenyl alcohols composition sold under the name Sensanov™ WR; soaps, for example sodium stearate or triethanolammonium stearate, or N-acylated derivatives of amino acids which are salified, for instance stearoyl glutamate.

As examples of emulsifying cationic surfactants optionally present in the composition (F) for topical use according to the invention, mention may be made of amine oxides, quaternium-82, cetyltrimethylammonium chloride, hexadecyltrimethylammonium bromide, cetylpyridinium chloride, benzalkonium chloride, benzethonium chloride, and the surfactants described in WO 96/00719 and mainly those in which the fatty chain comprises at least 16 carbon atoms.

As examples of opacifiers and/or nacreous agents optionally present in the pharmaceutical composition (F) for topical use according to the invention, mention may be made of sodium palmitate, sodium stearate, sodium hydroxystearate, magnesium palmitate, magnesium stearate, magnesium hydroxystearate, ethylene glycol monostea rate, ethylene glycol d istea rate, polyethylene glycol monostea rate, polyethylene glycol d istea rate, and fatty alcohols including 12 to 22 carbon atoms.

As examples of texturing agents optionally present in the pharmaceutical composition (F) for topical use according to the invention, mention may be made of N-acylamino acid derivatives, for example lauroyl lysine sold under the name Aminohope™ LL, octenyl starch succinate sold under the name Dryflo™, myristyl polyglucoside sold under the name Montanov 14, cellulose fibers, cotton fibers, chitosan fibers, talc, sericite and mica.

As examples of solvents and cosolvents optionally present in the pharmaceutical composition (F) for topical use according to the invention, mention may be made of water, organic solvents, for example glycerol, diglyceryl, glycerol oligomers, ethylene glycol, propylene glycol, butylene glycol, hexylene glycol, diethylene glycol, xylitol, erythritol, sorbitol, water-soluble alcohols such as ethanol, isopropanol or butanol, mixtures of water and of said organic solvents, propylene carbonate, ethyl acetate, benzyl alcohol and dimethyl sulfoxide (DMSO).

As examples of agents for improving the skin penetration optionally present in the pharmaceutical composition (F) for topical use according to the invention, mention may be made of glycol ethers, for instance ethylene glycol monomethyl ether, ethylene glycol monoethyl ether, ethylene glycol monopropyl ether, ethylene glycol monoisopropyl ether, ethylene glycol monobutyl ether, ethylene glycol monophenyl ether, ethylene glycol monobenzyl ether, diethylene glycol monomethyl ether, diethylene glycol monoethyl ether and diethylene glycol mono-n-butyl ether, diethylene glycol monoethyl ether (or Transcutol-P), fatty acids such as oleic acid, fatty acid esters of glycerol, for instance glyceryl behenate, glyceryl palm itostearate, behenoyl macroglycerides, polyoxyethylene-2-stearyl ether, polyoxyethylene-2-oleyl ether, terpenes, for instance D-limonene, and essential oils, for instance the essential oil of eucalyptus.

As examples of thickening and/or gelling agents optionally present in the pharmaceutical composition (F) for topical use according to the invention, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

As examples of thickening and/or gelling agents optionally present in the pharmaceutical composition (F) for topical use according to the invention, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, and glucosaminoglycans.

As examples of thickening and/or gelling agents optionally present in the pharmaceutical composition (F) for topical use according to the invention, mention may be made of cellulose, cellulose derivatives such as methyl cellulose, ethyl cellulose, hydroxypropyl cellulose, silicates, starch, hydrophilic starch derivatives, and polyurethanes.

As examples of stabilizers optionally present in the pharmaceutical composition (F) for topical use according to the invention, mention may be made of microcrystalline waxes, and more particularly ozokerite, and mineral salts such as sodium chloride or magnesium chloride.

As examples of thermal or mineral waters which can be combined with the pharmaceutical composition (F) for topical use according to the invention, mention may be made of thermal or mineral waters having a mineralization of at least 300 mg/l, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

As examples of active agents which can be combined with the pharmaceutical composition (F) for topical use according to the invention, mention may be made of substances or compositions which provide a beneficial effect to the human or animal subject.

These active agents may, for example, be antibodies, analgesics, anti-inflammatories, cytokines, cytoxins, growth factors, hormones, lipids, oligonucleotides, polymers, polysaccharides, polypeptides, protease inhibitors, vitamins, insect repellents, antibiotics or anti-inflammatory agents.

As examples of analgesic and anti-inflammatory agents that can be combined with the pharmaceutical composition (F) for topical use according to the invention, mention may be made of acetaminophen, aspirin, salicylic acid, methyl salicylate, choline salicylate, glycol salicylate, 1-menthol, camphor, mefenamic acid, fluphenamic acid, indomethacin, protizidic acid, fentiazac, tolmetin, tiaprofenic acid, phenylbutazone, oxyphenbutazone, clofezone, pentazocine, mepirizole, hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone and betamethasone.

As examples of non-steroidal anti-inflammatory agents (or NSAIDs) which can be combined with the pharmaceutical composition (F) for topical use according to the invention, mention may be made more particularly of arylacetic (or arylalkanoic) derivatives and 2-arylpropionic acids (or profens), and even more particularly diclofenac, tiaprofenic acid, alminoprofen, etodolac, flurbiprofen, ibuprofen, ketoprofen and naproxen.

As examples of antiseptic agents which can be combined with the pharmaceutical composition (F) for topical use according to the invention, mention may be made of cetrimide, povidone-iodine, chlorhexidine, iodine, benzalkonium chloride, benzoic acid, nitrofurazone, benzoyl peroxide, hydrogen peroxide, hexachlorophene, phenol, resorcinol and cetylpyridinium chloride.

As examples of antisectide agents which can be combined with the pharmaceutical composition (F) for topical use according to the invention, mention may be made of trichlorfone, triflumerone, fenthion, bendiocarb, cyromazine, dislubenzurone, dicyclanil, fluazurone, amitraz, deltamethrin, cypermethrin, chlorfenbinphose, flumethrin, ivermectin, abermectin, avermectin, doramectin, moxidectin, zeti-cypermethrin, diazinone, spinosad, imidacloprid, nitenpyran, pyriproxysene, sipronil, cythioate, lufenurone, selamectin, milbemycin oxime, chlorpyrifose, coumaphose, propetamphose, alpha-cypermethrin, highciscypermethrin, ivermectin, diflubenzurone, cyclodiene, carbamate and benzoyl urea.

As examples of antimicrobial agents which can be combined with the pharmaceutical composition (F) for topical use according to the invention, mention may be made of sulfonamides, aminoglycosides, for instance neomycin, tobramycin, gentamycin, amikacin, kanamycin, spectinomycin, paromomycin, netilmicin, polypeptides, cephalosporins, oxazolidinones, for instance ciprofloxacin, levofloxacin and ofloxacin.

As examples of active agents which can be combined with the pharmaceutical composition (F) for topical use according to the invention, mention may be made of vitamin E, Coenzyme Q10, L-carnitine, choline, folic acid, magnesium and salts thereof, caprylic acid, linoleic acid, lauric acid, taurine, vitamin C, vitamin A, and group B vitamins.

### EXAMPLES

The examples that follow illustrate the invention without, however, limiting it.

### Example 1: Preparation of a Concentrated Water-in-Oil Emulsion According to the Invention, Composed of C15-19 Alkanes as Fatty Phase and Sodium PGGA Crosslinked with 1,4-butanediol diglycidyl ether in Aqueous Phase (pH=5.5 to 6.0)

The synthetic process comprises the following steps:
Step a): Preparation of a sodium PGGA gel:
110 grams of demineralized water are placed under mechanical stirring provided by a Rayneri™ brand stirrer equipped with a deflocculator-type rotor.
30 grams of sodium PGGA sold under the brand name “Cosmetic Grade Sodium PolyGammaGlutamate” by the company Lubon are slowly added to the vortex.
Step b): Adjustment of the pH of the reaction medium to between 5.5 and 6 at a temperature of 20° C. using a 5M HCl solution.
Step c): Addition of 0.45 gram of 1,4-butanediol diglycidyl ether (sold under the name Erisys™ GE 21 by the company Emerald) to the aqueous phase prepared in step b)
Step d): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 20 grams of C15-19 alkane (sold under the name Emogreen™ L19 by the company SEPPIC)
Weigh out 30 grams of C11-12 isoparaffin (sold under the name Isopar H by the company ExxonMobil Chemical)

Homogenize the organic phase by mixing using a magnetic stirrer and a magnetic bar.
Step e): Pre-emulsification: Addition of the organic phase prepared in step d) to the aqueous phase prepared in step c) with mechanical stirring provided by a Rayneri™ brand stirrer equipped with a deflocculating-type rotor.
Step f): Shearing emulsification provided by a rotor-stator type system by a Silverson™ L4RT mixer for 2 minutes at a speed of 7500 rpm.
Step g): Vacuum distillation, in a reactor under a partial vacuum, of the light oil and the water.
Step h): Addition of an oil-in-water type surfactant to the concentrated emulsion obtained in step g): weighing out 8 grams of concentrated emulsion and addition of 2 grams of polyglyceryl-6 laurate.
Stirring the mixture to obtain a composition (E1).

### Example 2: Preparation of a Concentrated Water-in-Oil Emulsion According to the Invention, Composed of ethylhexyl palmitate as Fatty Phase and Sodium PGGA, Crosslinked with 1,4-butanediol diglycidyl ether in Aqueous Phase (pH=5.5 to 6.0)

The synthetic process comprises the following steps:
Step a): Preparation of a sodium PGGA gel:
120 grams of demineralized water are placed in a beaker with stirring provided by a Rayneri™ brand mechanical stirrer equipped with a deflocculator-type rotor.
20 grams of sodium PGGA, sold under the brand name Cosmetic Grade Sodium PolyGammaGlutamate by the company Lubon, are added slowly to the vortex.
Step b): Addition of 0.50 gram of 1,4-butanediol diglycidyl ether (sold under the name Erisys™ GE 21 by the company Emerald) to the aqueous phase prepared in step a)
Step c): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar H by the company ExxonMobil Chemical)

Homogenize the organic phase by mixing using a magnetic stirrer and a magnetic bar.
Step d): Pre-emulsification: Addition of the organic phase prepared in step c) to the aqueous phase prepared in step b) with mechanical stirring provided by a Rayneri™ brand stirrer equipped with a deflocculating-type rotor.
Step e): Shearing emulsification with a Silverson™ L4RT rotor-stator type device for 2 minutes at a speed of 7500 rpm.
Step f): Vacuum distillation, in a reactor under a partial vacuum, of the light oil and the water.
Step g): Addition of an oil-in-water type surfactant to the concentrated emulsion obtained in step f): weighing out 8 grams of concentrated emulsion obtained in step f) and addition of 2 grams of polyglyceryl-6 laurate.

The mixture is homogenized at room temperature with mechanical stirring at moderate speed to obtain composition (E2).

### Example 3: Preparation of a Concentrated Water-in-Oil Emulsion According to the Invention, Composed of a Mixture of ethylhexyl palmitate/C15-19 Alkanes as Fatty Phase and Sodium PGGA, Crosslinked with 1,4-butanediol diglycidyl ether in the Aqueous Phase (pH=5.5 to 6.0)

The synthetic process comprises the following steps:
Step a): Preparation of a sodium PGGA gel:
100 grams of demineralized water are placed in a beaker with stirring provided by a Rayneri™ brand mechanical stirrer equipped with a deflocculator-type rotor.
30 grams of PGGA sold under the brand name “Cosmetic Grade Sodium PolyGammaGlutamate” by the company Lubon are slowly added to the vortex.
Step b): Adjustment of the pH of the reaction medium to between 5.5 and 6 at a temperature of 20° C. using a 5M HCl solution.
Step c): Addition of 0.75 gram of 1,4-butanediol diglycidyl ether (sold under the name Erisys™ GE 21 by the company Emerald) to the aqueous phase prepared in step b)
Step d): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 10 grams of ethylhexyl palmitate (sold under the name Dub PO by the company Stéarinerie Dubois)
Weigh out 10 grams of C15-19 alkane (sold under the name Emogreen™ L19 by the company SEPPIC)
Weigh out 30 grams of C11-12 isoparaffin (sold under the name Isopar H by the company ExxonMobil Chemical)

The mixture obtained is stirred using a magnetic stirrer and a magnetic bar.
Step e): Pre-emulsification: Addition of the organic phase prepared in step d) to the aqueous phase prepared in step c) with mechanical stirring using a Rayneri™ brand stirrer equipped with a deflocculating-type rotor.
Step f): Emulsification by shear stirring with a rotor-stator type device with a Silverson™ L4RT stirrer for 2 minutes at 7500 rpm.
Step g): Vacuum distillation, in a reactor under a partial vacuum, of the light oil and the water.
Step h): Addition of an oil-in-water type surfactant to the concentrated emulsion obtained in step g): weighing out 8 grams of concentrated emulsion obtained in step g) and addition of 2 grams of polyglyceryl-6 laurate.

The mixture is homogenized at room temperature with mechanical stirring at moderate speed to obtain the composition (E3).

### Example 4: Preparation of a Concentrated Water-in-Oil Emulsion According to the Invention, Composed of ethylhexyl palmitate as the Fatty Phase and of Sodium PGGA Crosslinked with 1,4-butanediol diglycidyl ether in the Organic Phase (pH=5.5 to 6.0) (Crosslinking Agent in the Fatty Phase)

The synthetic process comprises the following steps:
Step a): Preparation of a sodium PGGA gel:
120 grams of demineralized water are placed in a beaker under mechanical stirring using a Rayneri™ brand stirrer equipped with a deflocculating type rotor.
20 grams of PGGA sold under the brand name Cosmetic Grade Sodium PolyGammaGlutamate by the company Lubon are slowly added to the vortex.
Step b): Adjustment of the pH of the reaction medium to between 5.5 and 6 at a temperature of 20° C. using a 4M NaOH solution.
Step c): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name DehymulsT™ PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar H by the company ExxonMobil Chemical)
Weigh out 0.50 gram of 1,4-butanediol diglycidyl ether (sold under the name Erisys GE 21 by the company Emerald)

Stir the mixture of ingredients previously weighed out with magnetic stirring using a magnetic bar.
Step d): Pre-emulsification: Addition of the organic phase prepared in step c) to the aqueous phase prepared in step b) with mechanical stirring using a Rayneri™ brand mechanical stirrer equipped with a deflocculating-type rotor.
Step e): Shearing emulsification with a stirrer equipped with a Silverson™ L4RT rotor-stator system for 2 min at a speed of 7500 rpm.
Step f): Vacuum distillation, in a reactor under a partial vacuum, of the light oil and the water.
Step g): Addition of oil-in-water surfactant to the concentrated emulsion obtained in step f): weighing out 8 grams of concentrated emulsion and 2 grams of polyglyceryl-6 laurate, which are added to the mixture obtained in step f).

The mixture is homogenized at room temperature with mechanical stirring at moderate speed to obtain composition (E4).

### Example 5: Preparation of a Concentrated Water-in-Oil Emulsion According to the Invention, Composed of ethylhexyl palmitate as Fatty Phase and Sodium PGGA, Crosslinked with 1,4-butanediol diglycidyl ether in the Aqueous Phase (pH=4)

The synthetic process comprises the following steps:
Step a): Preparation of a sodium PGGA gel:
120 grams of demineralized water are placed in a beaker with mechanical stirring using a Rayneri™ brand stirrer equipped with a deflocculating type rotor.
20 grams of PGGA sold under the brand name Cosmetic Grade Sodium PolyGammaGlutamate by the company Lubon are slowly added to the vortex.
Step b): Adjustment of the pH of the reaction medium to 4 at a temperature of 20° C. using a 5M HCl solution.
Step c): Addition of 0.50 gram of 1,4-butanediol diglycidyl ether (sold under the name Erisys™ GE 21 by the company Emerald) to the aqueous phase prepared in step b)
Step d): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar H by the company ExxonMobil Chemical)

Add each of the ingredients to the beaker and stir the mixture with a mechanical stirrer equipped with a magnetic bar.
Step e): Pre-emulsification: Addition of the organic phase prepared in step d) to the aqueous phase prepared in step c) with mechanical stirring using a Rayneri™ brand stirrer equipped with a deflocculating-type rotor.
Step f): Shearing emulsification with Silverson™ L4RT, for 2 minutes at a speed of 7500 rpm.
Step g): Vacuum distillation, in a reactor under a partial vacuum, of the light oil and the water.
Step h): Addition of water-in-oil surfactant in the concentrated emulsion obtained in step g): weighing out 8 grams of concentrated emulsion obtained in step g) and addition of 2 grams of polyglyceryl-6 laurate.

The mixture is homogenized at room temperature with mechanical stirring at moderate speed to obtain composition (E5).

### Example 6: Preparation of a Concentrated Water-in-Oil Emulsion According to the Invention, Composed of ethylhexyl palmitate as the Fatty Phase and of Sodium PGGA Crosslinked with 1,4-butanediol diglycidyl ether in the Aqueous Phase (pH=10)

The synthetic process comprises the following steps:
Step a): Preparation of a sodium PGGA gel:
120 grams of demineralized water are placed under mechanical stirring using a Rayneri™ brand stirrer equipped with a deflocculating type rotor.
20 grams of PGGA sold under the brand name Cosmetic Grade Sodium PolyGammaGlutamate by the company Lubon are slowly added to the vortex.
Step b): Adjustment of the pH of the reaction medium to 10 at a temperature of 20° C. using a 4M NaOH solution.
Step c): Addition of 0.50 gram of 1,4-butanediol diglycidyl ether (sold under the name Erisys™ GE 21 by the company Emerald) to the aqueous phase prepared in step b)
Step d): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar H by the company ExxonMobil Chemical)

Mix the various ingredients and stir the mixture using a magnetic stirrer equipped with a magnetic bar.
Step e): Pre-emulsification: Addition of the organic phase prepared in step d) to the aqueous phase prepared in step c) with mechanical stirring using a Rayneri™ brand stirrer equipped with a deflocculating-type rotor.
Step f): Shearing emulsification with a Silverson™ L4RT stirrer for 2 minutes at a speed of 7500 rpm.
Step g): Vacuum distillation (either with a rotavapor+flask or in a vacuum reactor) of the light oil and the water.
Step h): Addition of oil-in-water surfactant to the concentrated emulsion obtained in step g): weighing out 8 grams of concentrated emulsion obtained in step g) and 2 grams of polyglyceryl-6 laurate.

The mixture is homogenized at room temperature with mechanical stirring at moderate speed to obtain composition (E6).

### Example 7: Preparation of a Concentrated Water-in-Oil Emulsion According to the Invention, Composed of ethylhexyl palmitate as Fatty Phase and of Sodium PGGA Crosslinked with 1,4-butanediol diglycidyl ether and Lyophilized with C12-14 glycidyl ether in the Aqueous Phase (pH=?)

The synthetic process comprises the following steps:
Step a): Preparation of a sodium PGGA gel:
120 grams of demineralized water are placed in a beaker with stirring using a Rayneri™ brand stirrer equipped with a deflocculator-type rotor.
20 grams of PGGA sold under the brand name Cosmetic Grade Sodium PolyGammaGlutamate by the company Lubon are slowly added to the vortex.
Step b): Adjustment of the pH of the reaction medium to between 5.5 and 6 at a temperature of 20° C. using a 4M NaOH solution.
Step c): Addition of 0.5 gram of 1,4-butanediol diglycidyl ether (sold under the name Erisys™ GE 21 by the company Emerald) to the aqueous phase prepared in step b)
Step d): Addition of 2.0 grams of C12-C14 glycidyl ether (sold under the name Erisys™ GE 08 from the company Emerald) to the aqueous phase prepared in step c)
Step e): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar H by the company ExxonMobil Chemical)

Stir the mixture of ingredients with a magnetic stirrer equipped with a magnetic bar.
Step f): Pre-emulsification: Addition of the organic phase prepared in step e) to the aqueous phase prepared in step d) with mechanical stirring using a Rayneri™ brand stirrer equipped with a deflocculating-type rotor.
Step g): Shearing emulsification with a Silverson™ L4RT stirrer for 2 minutes at a speed of 7500 rpm.
Step h): Vacuum distillation, in a reactor under a partial vacuum, of the light oil and the water.
Step i): Addition of oil-in-water surfactants to the concentrated emulsion obtained in step g): weighing out 8 grams of concentrated emulsion obtained in step h) and 2 grams of polyglyceryl-6 laurate.

The mixture is homogenized at room temperature with mechanical stirring at moderate speed to obtain composition (E7).

### Example 8: Preparation of a Concentrated Water-in-Oil Emulsion According to the Invention, Composed of ethylhexyl palmitate as Fatty Phase and Sodium PGGA, Crosslinked with trimethylolethane triglycidyl ether in the Aqueous Phase (pH=6.0)

The synthetic process comprises the following steps:
Step a): Preparation of a sodium PGGA gel:
120 grams of demineralized water are placed under mechanical stirring with a Rayneri™ brand stirrer equipped with a deflocculator-type rotor.
20 grams of PGGA sold under the brand name Cosmetic Grade Sodium PolyGammaGlutamate by the company Lubon are slowly added to the vortex.
Step b): Adjustment of the pH of the reaction medium to between 5.5 and 6.0 at a temperature of 20° C. using a 4M NaOH solution.
Step c): Addition of 0.5 gram of trimethylolethane triglycidyl ether (sold under the name Erisys™ GE 31 by the company Emerald) to the aqueous phase prepared in step b)
Step d): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar™ H by the company ExxonMobil Chemical)

Stir the mixture of all the ingredients with a magnetic stirrer equipped with a magnetic bar.
Step e): Pre-emulsification: Addition of the organic phase prepared in step d) to the aqueous phase prepared in step c) with mechanical stirring using a Rayneri™ brand stirrer equipped with a deflocculating-type rotor.
Step f): Shearing emulsification with a Silverson™ L4RT stirrer for 2 minutes at a speed of 7500 rpm.
Step g): Vacuum distillation, in a reactor under a partial vacuum, of the light oil and the water.
Step h): Addition of oil-in-water surfactants to the concentrated emulsion obtained in step g): weighing out 8 grams of concentrated emulsion obtained in step f) and 2 grams of polyglyceryl-6 laurate.

The mixture is homogenized at room temperature with mechanical stirring at moderate speed to obtain composition (E8).

### Example 9: Preparation of a Crosslinked PGA(Na) Concentrated Inverse Latex According to the Invention in octyl palmitate

The synthetic process comprises the following steps:
Step a): Production of a sodium PGGA gel with a Rayneri™ brand mechanical stirrer equipped with a deflocculating-type rotor:
110 grams of demineralized water are placed in a beaker and stirred with a Rayneri™ brand mechanical stirrer equipped with a deflocculator-type rotor.
30 grams of PGGA sold under the brand name Cosmetic Grade Sodium PolyGammaGlutamate by the company Lubon are slowly added to the vortex.
Step b): Adjustment of the pH of the reaction medium to 5.5-6 at a temperature of 20° C. using a 5M HCl solution.
Step c): Addition of 0.72 gram of 1,4-butanediol diglycidyl ether (sold under the name Erisys™ GE 21 by the company Emerald) to the aqueous phase prepared in step b)
Step d): Preparation of the organic phase in a 100 gram beaker:
Weigh out 5 grams of sorbitan isostearate (sold under the name Montane™ 70 VG by the company SEPPIC)
Weigh out 3 grams of a mixture consisting of tall-oil diethanolamide sold under the brand name Simaline™ IE 200 by the company SEPPIC.
Weigh out 2 grams of a polymeric surfactant sold under the brand name Hypermer™ 6212 by the company Croda
Weigh out 50 grams of C11-12 isoparaffin (sold under the name Isopar™ H by the company ExxonMobil Chemical)

Stir the mixture of all the ingredients with a magnetic stirrer equipped with a magnetic bar.
Step e): Pre-emulsification: Addition of the organic phase prepared in step d) to the aqueous phase prepared in step c) with mechanical stirring using a Rayneri™ brand stirrer equipped with a deflocculating-type rotor.
Step f): Shearing emulsification with a Silverson™ L4RT mechanical stirrer for 2 minutes at a speed of 7500 rpm.
Step g): Vacuum distillation, in a reactor under a partial vacuum, of the light oil and the water.
Step h): Addition of oil-in-water surfactant to the concentrated emulsion obtained in step g): weighing out 8 grams of concentrated emulsion and 1 gram of polysorbate 80 (sold under the name Montanox™ 80 by the company SEPPIC.

The mixture is homogenized at room temperature with mechanical stirring at moderate speed to obtain composition (E9).

### Example 10: Preparation of a Concentrated Water-in-Oil Emulsion According to the Invention, Composed of ethylhexyl palmitate as the Fatty Phase and of Sodium PGGA Crosslinked with ethylene glycol diglycidyl ether (EGDGE) in the Aqueous Phase (pH=6.0)

The synthetic process comprises the following steps:
Step a): Preparation of a sodium PGGA gel:
130 grams of demineralized water are placed under mechanical stirring with a Rayneri™ brand stirrer equipped with a deflocculator-type rotor.
10 grams of PGGA sold under the brand name Cosmetic Grade Sodium PolyGammaGlutamate by the company Lubon are slowly added to the vortex.
Step b): Adjustment of the pH of the reaction medium to between 5.5 and 6 at a temperature of 20° C. using a 4M NaOH solution.
Step c): Addition of 0.25 gram of ethylene glycol diglycidyl ether (sold under the name Erisys™ EGDGE by the company Emerald) to the aqueous phase prepared in step b)
Step d): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar™ H by the company ExxonMobil Chemical)

Stir the mixture consisting of all the above ingredients with a magnetic stirrer equipped with a magnetic bar.
Step e): Pre-emulsification: Addition of the organic phase prepared in step d) to the aqueous phase prepared in step c) with mechanical stirring using a Rayneri™ brand stirrer equipped with a deflocculating-type rotor.
Step f): Shearing emulsification with a Silverson™ L4RT stirrer for 2 minutes at a speed of 7500 rpm.
Step g): Vacuum distillation, in a reactor under a partial vacuum, of the light oil and the water.
Step h): Addition of oil-in-water surfactants to the concentrated emulsion obtained in step f): weighing out 8 grams of concentrated emulsion obtained in step g) and 2 grams of polyglyceryl-6 laurate.

The mixture is homogenized at room temperature with mechanical stirring at moderate speed to obtain composition (E10).

### Example 11: Preparation of a Concentrated Water-in-Oil Emulsion According to the Invention, Composed of ethylhexyl palmitate as Fatty Phase and of Sodium PGGA Crosslinked with 1,4-butanediol diglycidyl ether in the Aqueous Phase (pH=6.0)

The synthetic process comprises the following steps:
Step a): Preparation of a sodium PGGA gel:
100 grams of demineralized water are placed under mechanical stirring with a Rayneri™ brand stirrer equipped with a deflocculator-type rotor.
40 grams of PGGA sold under the brand name Cosmetic Grade Sodium PolyGammaGlutamate by the company Lubon are slowly added to the vortex.
Step b): Adjustment of the pH of the reaction medium to between 5.5 and 6 at a temperature of 20° C. using a 4M NaOH solution.
Step c): Addition of 0.80 gram of 1,4-butanediol diglycidyl ether (sold under the name Erisys™ GE 21 by the company Emerald) to the aqueous phase prepared in step b)
Step d): Preparation of the organic phase in a 100 gram beaker:
Weigh out 5 grams of sorbitan oleate (sold under the name Montane™ 80 VG by the company SEPPIC)
Weigh out 5 grams of polyglyceryl-2 dipolyhydroxystearate (sold under the name Dehymuls™ PGPH by the company BASF)
Weigh out 20 grams of ethylhexyl palmitate (sold under the name Dub PO by the company Stéarinerie Dubois)
Weigh out 30 grams of C11-12 isoparaffin (sold under the name Isopar™ H by the company ExxonMobil Chemical)

Stir the mixture consisting of all the weighed out ingredients with a magnetic stirrer equipped with a magnetic bar.
Step e): Pre-emulsification: Addition of the organic phase prepared in step d) to the aqueous phase prepared in step c) with mechanical stirring using a Rayneri™ brand stirrer equipped with a deflocculating-type rotor.
Step f): Shearing emulsification with a Silverson L4RT stirrer for 2 minutes at a speed of 7500 rpm.
Step g): Vacuum distillation, in a reactor under a partial vacuum, of the light oil and the water.
Step h): Addition of oil-in-water surfactants to the concentrated emulsion obtained in step g): weighing of 8 grams of concentrated emulsion obtained in step g) and 2 grams of Polyglyceryl-6 laurate. Stirring

[symbol]

The mixture is homogenized at room temperature with mechanical stirring at moderate speed to obtain composition (E11).

Evaluation of compositions (E1) to (E11) according to the invention. The evaluation of the compositions (E1) to (E11) according to the invention is performed as described below:
Weigh out 192 grams of water in a 400 ml high-sided beaker.
Add with mechanical stirring, using a Rayneri brand stirrer equipped with a deflocculating-type rotor device, 8 grams of compositions (E1) to (E11).
Leave stirring until a homogeneous gel is obtained.
Measure the dynamic viscosity of the homogeneous gels using a Brookfield RVT brand viscometer, at a speed of 5 rpm, choosing the appropriate spindle.
Add 0.1% by mass of sodium chloride to the gel previously obtained, and stir with a Rayneri brand mechanical stirrer equipped with a deflocculator-type rotor.
Then measure the dynamic viscosity of such a new gel using a Brookfield RVT brand viscometer at a speed of 5 rpm, choosing the appropriate spindle.

The results are collated in Table 1 below.

TABLE 1

| | | | | Gel viscosity |
|---|---|---|---|---|
| | | Gel viscosity 4% by | | obtained in step a) |
| | Gel viscosity 4% by | mass of composition + | Equivalent | of the preparation |
| | mass of composition | 0.1% by mass NaCl | mass % of | process |
| | Brookfield ™ RVT | Brookfield ™ RVT | polymeric | Brookfield ™ RVT |
| | speed 5 | speed 5 | active | speed 5 |
| Composition | Spindle (x) | Spindle (x) | material | Spindle (x) |
| Control Test | 2% gel = 176 mPa · s | 2% gel + 0.1% NaCl = | 2% | 2% gel = 128 mPa · s |
| “Cosmetic grade | (Spindle 2) | 128 mPa · s | | (Spindle 2) |
| sodium PGGA” | | (Spindle 2) | | |
| sold by the | | | | |
| company Lubon | | | | |
| Composition(E₁) | 76 200 mPa · s | 73 000 mPa · s | 2% | 18 120 mPa · s |
| | (Spindle 6) | (Spindle 6) | | (Spindle 3) |
| Composition(E₂) | 124 200 mPa · s | 89 400 mPa · s | 1.6% | 5040 mPa · s |
| | (Spindle 6) | (Spindle 6) | | (Spindle 3) |
| Composition(E₃) | 91 600 mPa · s | 816 mPa · s | 2% | 18 120 mPa · s |
| | (Spindle 6) | (Spindle 3) | | (Spindle 3) |
| Composition(E₄) | 117 200 mPa · s | 58 600 mPa · s | 1.6% | 5040 mPa · s |
| | (Spindle 6) | (Spindle 6) | | (Spindle 3) |
| Composition(E₅) | 9820 mPa · s | 8860 mPa · s | 1.6% | 5040 mPa · s |
| | (Spindle 3)* | (Spindle 6)* | | (Spindle 3) |
| Composition(E₆) | 131 800 mPa · s | 102 800 mPa · s | 1.6% | 5040 mPa · s |
| | (Spindle 6)* | (Spindle 6)* | | (Spindle 3) |
| Composition(E₇) | 167 600 mPa · s | 118 400 mPa · s | 1.6% | 5040 mPa · s |
| | (Spindle 6) | (Spindle 6) | | (Spindle 3) |
| Composition(E₈) | 9540 mPa · s | 8860 mPa · s | 1.6% | 5040 mPa · s |
| | (Spindle 3) | (Spindle 3) | | (Spindle 3) |
| Composition(E₉) | 91 800 mPa · s | 34 600 mPa · s | 1.33% | 18 120 mPa · s |
| | (Spindle 6) | (Spindle 6) | | (Spindle 3) |
| Composition(E₁₀) | 78 000 mPa · s | 600 mPa · s | 1% | 900 mPa · s |
| | (Spindle 6) | (Spindle 2) | | (Spindle 3) |
| Composition(E₁₁) | 74 800 mPa · s | 48 400 mPa · s | 2.28% | 50 000 mPa · s |
| | (Spindle 6) | (Spindle 6) | | (Spindle 6) |

1. A pharmaceutical composition comprising at least one pharmaceutical active principle and as thickener a composition in the form of an emulsion of the self-invertible water-in-oil type comprising, per 100% of its mass, a mass content of greater than or equal to 20% of a polymer (P) consisting of monomer units derived from partially or totally salified glutamic acid, and of units derived from at least one crosslinking agent bearing at least two glycidyl functions.

2. The pharmaceutical composition as claimed in claim 1, wherein, in composition, the mass content of the polymer is greater than or equal to 20% and less than or equal to 60%.

3. The pharmaceutical composition as claimed in claim 1, wherein, in composition, the crosslinking agent is chosen from the members of the group consisting of:
ethylene glycol diglycidyl ether of formula (I) 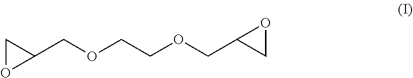 [SMILES: C(COCC1CO1)OCC1CO1]
the compound of formula (II) 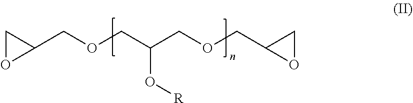
with R representing a hydrogen atom or the radical 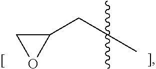 [SMILES: *CC1CO1]
and n which represents an integer greater than or equal to one and less than or equal to 10;
1,3-propanediol diglycidyl ether of formula (III) 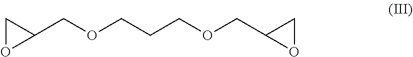 [SMILES: C(COCC1CO1)COCC1CO1]
1,2-propanediol diglycidyl ether of formula (IV) 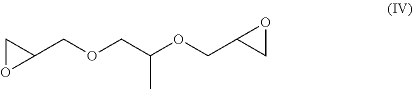
1,4-butanediol diglycidyl ether of formula (V) 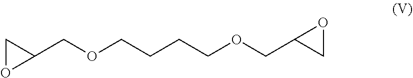 [SMILES: C(CCOCC1CO1)COCC1CO1]
1,2-butanediol diglycidyl ether of formula (VI) 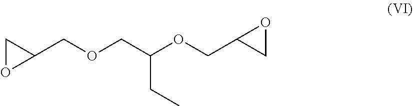
1,3-butanediol diglycidyl ether of formula (VII) 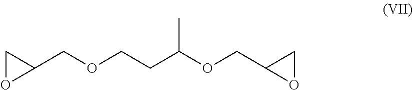
1,6-hexanediol diglycidyl ether of formula (VIII) 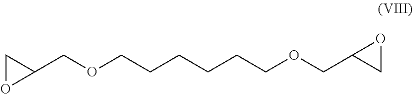 [SMILES: C(CCCOCC1CO1)CCOCC1CO1]
the compound of formula (IX) 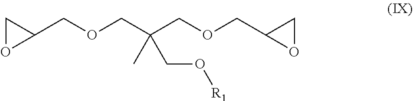
with R1 representing a hydrogen atom or the radical 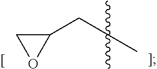 [SMILES: *CC1CO1]
when R1 represents a hydrogen atom, the compound of formula (IX) is more particularly the compound of formula (IXa) or trimethylolethane diglycidyl ether 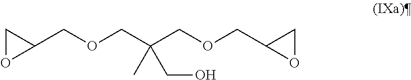 [SMILES: CC(CO)(COCC1CO1)COCC1CO1]
when R1 represents 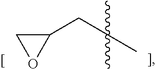 [SMILES: *CC1CO1]
the compound of formula (IX) is more particularly the compound of formula (IXb) or trimethylolethane triglycidyl ether 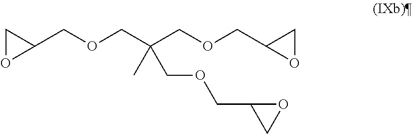 [SMILES: CC(COCC1CO1)(COCC1CO1)COCC1CO1]
the compound of formula (X) 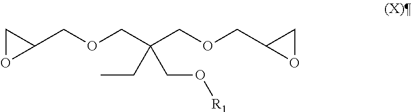
with R1 representing a hydrogen atom or the glycidyl radical 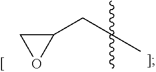 [SMILES: *CC1CO1]
when R1 represents a hydrogen atom, the compound of formula (X) is more particularly the compound of formula (Xa) or trimethylolpropane diglycidyl ether 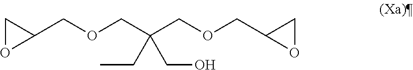 [SMILES: CCC(CO)(COCC1CO1)COCC1CO1]
when R1 represents the glycidyl radical 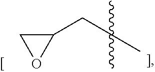 [SMILES: *CC1CO1]
the compound of formula (X) is more particularly the compound of formula (Xb) or trimethylolpropane triglycidyl ether 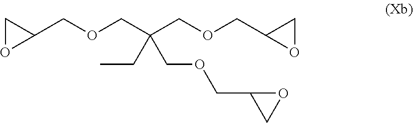 [SMILES: CCC(COCC1CO1)(COCC1CO1)COCC1CO1]
the compound of formula (XI) 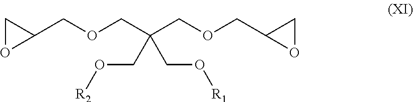
with R1 and R2, independent, which represent a hydrogen atom or the glycidyl radical 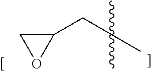 [SMILES: *CC1CO1]
when R1 and R2 each represent a hydrogen atom, the compound of formula (XI) is more particularly the compound of formula (XIa) or pentaerythrityl diglycidyl ether 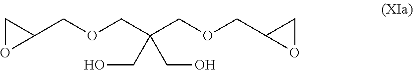 [SMILES: OCC(CO)(COCC1CO1)COCC1CO1]
when R1 represents a hydrogen atom and R2 represents the glycidyl radical 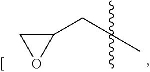 [SMILES: *CC1CO1]
the compound of formula (XI) is more particularly the compound of formula (XIb) or pentaerythrityl triglycidyl ether 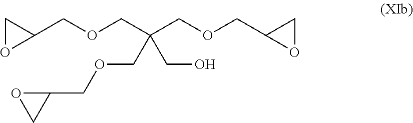 [SMILES: OCC(COCC1CO1)(COCC1CO1)COCC1CO1]
when R1 and R2 each represent the glycidyl radical 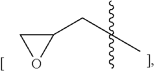 [SMILES: *CC1CO1]
the compound of formula (XI) is more particularly the compound of formula (XIc) or pentaerythrityl tetraglycidyl ether 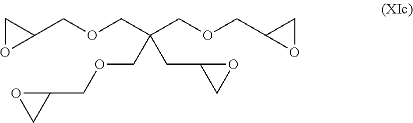 [SMILES: C(OCC(COCC1CO1)(COCC1CO1)CC1CO1)C1CO1]
the compound of formula (XII) 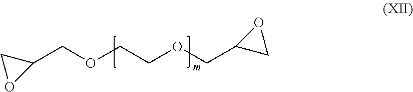
with m representing an integer greater than or equal to 2
the compound of formula (XIII) 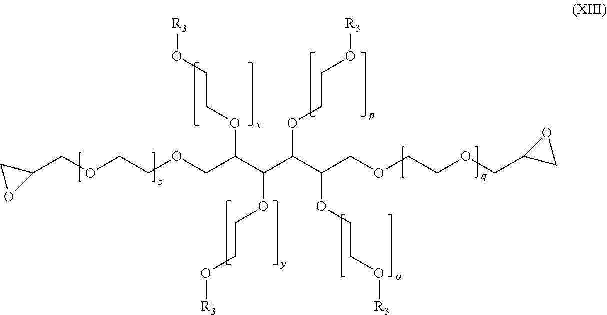
with R3 representing a hydrogen atom or 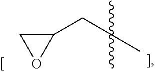 [SMILES: *CC1CO1]
and x, y, z, o, p and q, independently of each other, represent an integer greater than or equal to 2 and less than or equal to 10.

4. The pharmaceutical composition as claimed in claim 1, wherein, in composition, the polymer is gamma-polyglutamic acid in acid form, or partially or totally salified form.

5. The pharmaceutical composition as claimed in claim 1, wherein, in composition, the polymer, per 100 mol % of monomer units derived from partially or totally salified glutamic acid, the crosslinking agent represents from 0.5 mol % to 20 mol %.

6. The pharmaceutical composition as claimed in claim 1, wherein composition has a viscosity of between 100 mPa·s and 10000 mPa·s.

7. The composition as claimed in claim 1, wherein it also comprises a monomer unit derived from the compound of formula (X′): 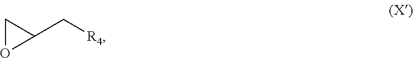
with R4 representing a linear or branched, saturated or unsaturated, functionalized or non-functionalized hydrocarbon-based radical including from 6 to 22 carbon atoms.

8. The pharmaceutical composition as claimed in claim 1, wherein it comprises between 0.1% and 10% by mass of said composition.

9. The pharmaceutical composition as claimed in claim 1, wherein the pharmaceutical active principle is chosen from antibacterial agents, antimicrobial agents, antiparasitic agents, antihelminthic agents, anticoccidial agents, anti-cryptosporidian agents, anti-protozoal agents, antimycotic agents, non-steroidal anti-inflammatory agents, antiallergic and immunomodulatory agents, analgesic agents, antihistamine agents, local anesthetic agents, antisecticidal agents, antiseptic agents and antifungal agents.

10. A process for preparing a pharmaceutical composition as defined in claim 1, comprising:
a step A) of preparing composition, comprising the following substeps:
a) preparation of an aqueous solution comprising partially or totally salified polyglutamic acid with said aqueous solution comprising, per 100% of its mass, between 5% and 70% by mass of partially or totally salified PGA and a crosslinking agent comprising at least two glycidyl functions,
b) adjustment of the pH of the aqueous solution obtained in step a) to a pH of between 3 and 11;
c) preparation of an organic phase containing at least one volatile oil, at least one other nonvolatile oil and at least one emulsifying surfactant of the water-in-oil type;
d) pre-emulsification by adding the organic phase obtained in step c) to the aqueous solution obtained in step b) with stirring;
e) emulsification of the pre-emulsion obtained in step d) by homogenization with stirring;
f) distillation of the water and volatile oil contained in the emulsion obtained in step e);
g) addition of at least one emulsifying surfactant of the oil-in-water type so as to obtain the composition; and,
a step B) of mixing at least one composition prepared in step A) with at least one pharmaceutical active principle and at least one pharmaceutically acceptable medium.

11. The process as claimed in claim 10, wherein, in step a), the polyglutamic acid is gamma-polyglutamic acid.

12. The process as claimed in claim 11, wherein, in step a), all of the monomer units constituting the gamma-polyglutamic acid are derived from sodium glutamate, potassium glutamate, ammonium glutamate, calcium glutamate, magnesium glutamate or a mixture of these forms.

13. The process as claimed in claim 10, wherein, in step a), the crosslinking agent is present in mass proportions of between 0.5% and 10% by mass relative to the mass of polyglutamic acid.

14. The process as claimed in claim 13, wherein the crosslinking agent is chosen from the members of the group consisting of the compounds of formulae (I), (II), (IIa), (IIb), (IIc), (III), (IV), (V), (VI), (VII), (VIII), (IX), (IXa), (IXb), (X), (Xa), (Xb), (XI), (XIa), (XIb), (XIc), (XII) and (XIII)

15. The process as claimed in claim 10, wherein, in step c), the at least emulsifying agent of the water-in-oil type is chosen from the elements of the group consisting of sorbitan esters, polyglycerol esters, alkoxylated polyglycerol esters, polyglycol polyhydroxystearates, polyglyceryl polyhydroxystearates and alkoxylated polyglyceryl polyhydroxystearates.

16. The process as claimed in claim 10, wherein, in step c), the organic solution comprises, per 100% of its own mass, between 10% and 30% by mass of at least one emulsifying agent of the water-in-oil type, preferably between 15% and 20% by mass.

17. The process as claimed in claim 10, wherein, in step c), the emulsifying agent of the water-in-oil type is a polyglyceryl polyhydroxystearate.

18. The process as claimed in claim 10, wherein, in step g), the at least emulsifying surfactant of the oil-in-water type is chosen from members of the group consisting of a polyethoxylated fatty alcohol, a polyethoxylated hexitan ester, an alkylpolyglycoside, a composition of alkylpolyglycoside and of fatty alcohols, a polyglycerol ester, a composition of polyglycerol ester and of polyglycerol.

19. The use of said composition as defined in claim 1, as a thickening and/or emulsifying and/or stabilizing agent for a liquid aqueous pharmaceutical composition for topical use.
